(19) United States
(12) Patent Application Publication
(10) Pub. No.: US 2026/0201471 A1
(43) Pub. Date: Jul. 16, 2026

(54) METHODS OF ASSESSING A WOUND'S RESPONSIVENESS TO SPECIFIC TREATMENTS

(71) Applicant: Drexel University, Philadelphia, PA (US)

(72) Inventors: Kara L. Spiller, Glenside, PA (US); Jessica M. Eager, Philadelphia, PA (US); Ahmet Sacan, Media, PA (US)

(21) Appl. No.: 19/133,923

(22) PCT Filed: Nov. 30, 2023
(86) PCT No.: PCT/US2023/081737

§ 371 Date: May 29, 2025

Related U.S. Application Data
(60) Provisional application No. 63/429,676, filed on Dec. 2, 2022.

(51) Int. Cl. C12Q 1/6883; G06N 20/00; G16H 20/10; G16H 50/20

(52) U.S. Cl. CPC C12Q 1/6883; G06N 20/00; G16H 20/10; G16H 50/20; C12Q 2600/158

(57) ABSTRACT

Described herein is method of treating and/or ameliorating a wound in a subject in need thereof. The method includes measuring an expression of a first and second panel of genes from a sample from the wound, wherein when at least one gene from the first panel is expressed, a pro-inflammatory agent is administered to the subject, and wherein when at least one gene from the second panel is expressed, an anti-inflammatory or M2 macrophage-promoting agent is administered to the subject, thus treating and/or ameliorating the wound in the subject.

### CROSS-REFERENCE TO RELATED APPLICATIONS

This application claims priority under 35 U.S.C. § 119 (e) to U.S. Provisional Patent Application No. 63/429,676, filed October Dec. 2, 2022, which is hereby incorporated herein by reference in its entirety.

### BACKGROUND OF THE INVENTION

Diabetic foot ulcers (DFUs) continue to be a major complication for diabetic patients. They occur in approximately 15% of patients and often lead to lower extremity amputations, which in turn increase the 5-year mortality rate to upwards of 55%. One retrospective cohort analysis found that just 35% of DFUs heal within a year, and estimated average healing times of longer than 4 months. Neuropathy, poor limb perfusion, infection, epigenetic alterations, aging, and failure to comply with offloading instructions are associated with poor outcomes, but even under the best conditions, DFUs still fail to heal at an alarming rate. A particularly frustrating aspect of chronic wound care is that some wounds respond to treatment, while others do not, with no clear reasons for the heterogeneity in patient responsiveness. In general, the mechanisms behind impaired healing are poorly understood. While investigations into immune cell behavior using animal models have been instrumental to advancement of mechanistic understanding, diabetic animal models are notoriously inadequate for the study of chronic wounds, especially because they fail to replicate the heterogeneous nature of the human response to treatment in which some wounds never heal. As such, there remains a need for human clinical research to increase understanding of maladaptive immune cell function in healing and non-healing DFUs to better understand this heterogeneity so that more targeted therapeutics can be developed.

### BRIEF SUMMARY

In one aspect, the present disclosure generally relates to a method of treating and/or ameliorating a non-healing wound in a subject in need thereof, the method comprising measuring the levels of expression of a first panel of inflammation-related genes from a sample from the wound, and measuring the levels of expression of a second panel of inflammation-related genes from the sample, wherein when at least one gene from the first panel or a composite score of genes from the first panel or a ratio compared to genes from the second panel is expressed at a lower level than a reference sample, a pro-inflammatory agent is administered to the subject, and wherein when at least one gene from the second panel or a composite score of genes from the second panel or a ratio compared to genes from the first panel is expressed at a higher level than a reference sample, an anti-inflammatory or M2-promoting agent is administered to the subject, thus treating and/or ameliorating the wound in the subject. In some aspects, the method of embodiment 1, wherein the first panel and/or the second panel comprises RIMS2, CXCL11, EBI3, ST8SIA6, IFNG, IL6, IL3RA, FCGR2B, TLR2, SPP1, IL15, and TNFRSF1B. In some aspects, the first panel and/or the second panel comprises ADORA2A, ANKRD22, APOBEC3A, APOL1, ASPHD2, C1ORF61, CASP1, CCL1, CCL19, CCL5, CCL8, CCR7, CD38, CD80, CFB, CFH, CLEC4D, CLEC4E, CMPK2, CRISPLD2, CSF3, CXCL10, CXCL11, CXCL9, EBI3, EPHA2, FBXO2, GBP1, GBP4, GBP5, GCH1, HAPLN3, HCAR3, HLA-DOA, HLA-DOB, HSH2D, IDO1, IDO2, IFI44L, IFITM1, IFITM3, IGFBP4, IL15, IL15RA, ILIB, IL27, IL32, IL3RA, IL6, IL8, IRF1, ISG15, ISG20, ITK, KRT7, LAG3, MN1, MTIM, NCF1, NCF1B, NEURL3, NNMT, NOD2, OASL, PDE4B, PRSS8, PTGES, PTGS2, RIMS2, RSAD2, SERPINB7, SERPING1, TNFAIP6, TNIP3, UBD, VEGFA, XAF1, and ZBP1. In some aspects, the ratio compared to genes from the first panel or the ratio compared to genes from the second panel is a ratio of C3AR1 to CCL22, a ratio of RIMS2 to SIGLEC12, or a combination of a ratio of C3AR1 to CCL22 and a ratio of RIMS2 to SIGLEC12.

Furthermore, in one aspect, the present disclosure generally relates to a method of treating and/or ameliorating a non-healing wound in a subject in need thereof, the method comprising: measuring the levels of expression of a first panel of inflammation-related genes from a sample from the wound, and measuring the levels of expression of a second panel of M2 macrophage-related genes from the sample, wherein when the sample is classified by a previously-trained machine-learning algorithm as hypo-inflammatory, a pro-inflammatory agent is administered to the subject, and wherein when the sample is classified by a previously-trained machine-learning algorithm as hyper-inflammatory, an anti-inflammatory or M2-promoting agent is administered to the subject thus treating and/or ameliorating the wound in the subject. In some aspects, the second panel of M2 macrophage-related genes comprises ABCG2, ALDH1A2, ALOX15, ATOH8, CABLES1, CACNA1G, CACNB4, CCL22, CCL26, CCL28, CCDC85C, CD1C, CDH1, CH25H, CHDH, CLEC4G, COL5A3, CR2, CRB2, DACT1, DNASE1L3, DUOX1, DUOXA1, EHF, ENHO, FABP4, FAM110B, FCGR2B, FOXQ1, GCNT3, IL17RB, IL21R, LIMA1, LRRC4, MAOA, MEST, MORC4, MRC1, MS4A6E, MUCL1, NEK10, NIPAL1, OLFML3, PALD1, PCSK1, PDGFB, PLCB1, PLEKHA6, RAMP1, S100A1, SEMA3G, SIGLEC12, SLC25A48, SNAI3, ST8SIA6, SYT17, TAL1, TGM2, TIMP3, TNFRSF11A, TSPAN7, VTN, WDR66, WNT5B. In some aspects, the pro-inflammatory agent is selected from the group consisting of a glutaraldehyde-crosslinked material, a xenogeneic protein-derived material, a skin regeneration system, an acellular dermal matrix, a biologic extracellular matrix, biomaterials that promote the M1 phenotype in host macrophages through the release of drugs, proteins, or nucleic acids, and M1 macrophage cell therapy. In some aspects, the anti-inflammatory or M2-promoting agent is selected from the group consisting of an amniotic membrane-derived tissue, a placental-derived tissue, a bioengineered allogeneic cellular construct, an extracellular matrix-derived material, urinary bladder matrix (UBM), biomaterials that promote the M2 phenotype in host macrophages through the release of drugs, proteins, or nucleic acids, and M2 macrophage cell therapy. In some aspects, the wound is an ulcer. In some aspects, the wound is a diabetic ulcer. In some aspects, the sample is obtained by swabbing the wound, or debriding the wound and collecting the debrided tissue. In some aspects, expression is measured at the mRNA level or protein level. In some aspects, the method further comprises administering an additional treatment. In some aspects, the additional treatment is selected from the group consisting of debriding the wound, applying a compression wrapping, applying a compression stocking, applying dressings promoting a moist environment to the wound, applying a wound offloading device, applying a hyperbaric oxygen therapy, applying an antibiotic, administering an immunomodulation medication, or combinations thereof. In some aspects, the subject is a human.

Moreover, in one aspect, the present disclosure generally relates to a method of treating and/or ameliorating a non-healing wound in a subject in need thereof, the method comprising: measuring the levels of expression of a first panel of inflammation-related genes from a sample from the wound, and measuring the levels of expression of a second panel of inflammation-related genes from the sample, wherein when the sample is classified by a previously-trained machine-learning algorithm as hypo-inflammatory, a pro-inflammatory agent is administered to the subject, and wherein when the sample is classified by a previously-trained machine-learning algorithm as hyper-inflammatory, an anti-inflammatory or M2-promoting agent is administered to the subject, thus treating and/or ameliorating the wound in the subject. In some aspects, the previously-trained machine-learning algorithm is a Partial Least-Squares Discriminant Analysis (PLS-DA) algorithm, support vector machine, or neural network.

Moreover, in one aspect, the present disclosure generally relates to a method of treating and/or ameliorating a non-healing wound in a subject in need thereof, the method comprising: measuring the levels of expression of a panel of M2 macrophage-associated genes in a subject, wherein when the sample is classified by a previously-trained machine-learning algorithm as hyper-inflammatory or having too little M2 macrophage-associated gene expression, an anti-inflammatory or M2-promoting agent is administered to the subject, thus treating and/or ameliorating the wound in the subject. In some aspects, the previously-trained machine-learning algorithm is a Partial Least-Squares Discriminant Analysis (PLS-DA) algorithm, support vector machine, or neural network.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1A: Hierarchical clustering of DEGs between healing and non-healing DFUs.

FIG. 1B: Significantly differentially expressed genes in non-healing DFUs compared to healing DFUs at week 0. Welch's t-test and log FC>|1.5|.

FIG. 1C: Significantly differentially expressed genes in non-healing DFUs compared to healing DFUs at week 4 following initial sample collection/analysis.

FIG. 1D: PLS-DA of DEGs showing differences between healing and non-healing DFUs, without discriminating between non-healing subtypes.

FIG. 1E: Coefficients of variation, determined from PLS-DA of DEGs, showing genes useful for discriminating between healing and non-healing DFUs, without discriminating between non-healing subtypes.

FIG. 1F: PLS-DA of DEGs showing differences between two non-healing subtypes.

FIG. 1G: Coefficients of variation, determined from PLS-DA of DEGs between healing and non-healing DFUs, showing genes useful for discriminating between two non-healing subtypes.

FIG. 2: PLS-DA using entire 227-gene dataset showing separation between healing DFUs (middle), non-healing subtype 1 (right), and non-healing subtype 2 (left).

FIG. 3: PLS-DA using entire 227-gene dataset showing separation between two non-healing (NH) subtypes.

FIG. 4: Coefficient of variation from PLS-DA using entire 227-gene dataset to determine genes useful for distinguishing between non-healing subtypes (NH subtype 2-left; NH subtype 1-right).

FIG. 5: Variables important for projection (VIP) from PLS-DA using entire 227-gene dataset to determine genes that are useful for distinguishing between non-healing subtypes.

FIG. 6: Example genes distinguishing between non-healing subtype 2 compared to non-healing subtype 1 and healing DFUs.

FIG. 7: PLS-DA using entire 227-gene dataset showing separation between nonhealing subtype 1 and healing DFUs.

FIG. 8: Coefficients of variation from PLS-DA using entire 227-gene dataset showing genes that are useful for distinguishing between nonhealing subtype 1 and healing DFUs.

FIG. 9: Variables important for projection (VIP) from PLS-DA using entire 227-gene dataset to determine genes that distinguish between nonhealing subtype 1 and healing DFUs.

FIG. 10: Example genes that distinguish between nonhealing subtype 1 and healing DFUs. Most of these genes are pro-inflammatory or M1 macrophage markers, and since they are lower in nonhealing subtype 1 compared to healing, these results suggest that nonhealing subtype 1 are hypo-inflammatory.

FIG. 11: X-variate PLS-DA scores that distinguish between nonhealing subtypes.

FIG. 12: ROC curve showing sensitivity and specificity of using PLS-DA x-variate scores to predict non-healing outcome.

FIG. 13 presents an experimental design overview diagram. Debrided DFU tissue samples were collected and analyzed via NanoString for a panel of 227 inflammation-related genes and via 16S rRNA sequencing for microbial analysis. For some subjects, paired samples were collected after 3-4 weeks as a second time point for NanoString analysis.

FIG. 14 presents results related to gene expression patterns in healing and non-healing wounds.

FIG. 15A-FIG. 15E present results related to healing and non-healing wound subtypes. FIG. 15A presents the top 20 genes with the highest correlations of covariates identified from PLS-DA. One-way ANOVA with Tukey's multiple comparisons tests. FIG. 15B presents the top 4 genes with the highest correlations of covariates identified from PLS-DA. One-way ANOVA with Tukey's multiple comparisons tests. FIG. 15C presents PLS-DA scores for healing vs. non-healing subtype 1. FIG. 15D presents the top 20 genes with the highest correlations of covariates.

FIG. 15E presents results related to UBD, IL15, IL6, IL13RA, CCL8 and TSPAN7.

FIG. 16 presents graphical representations of the differences between groups of the top 20 genes of FIG. 15D.

FIG. 17 presents a graphical representation of results related to UBD, IL6, IL3RA, CCL8, CSF3, HLA-DOA, CXCL10, and CXCL9.

FIG. 18A-FIG. 18F present results related to gene expression in healing and non-healing wound types. FIG. 18A presents a graphical representation of CCL1 expression at enrollment.

FIG. 18B presents a graphical representation of the fold change (FC) of CCL1 expression in paired samples at week 4 vs. week 0, Welch's t-test. FIG. 18C presents a graphical representation of receiver operating characteristics (ROC) curve showing sensitivity and specificity of the fold change in CCL1 over 4 weeks for predicting healing outcome. FIG. 18D presents graphical representations of fold change in paired samples in ssGSEA scores for week 4 vs. week 0 of M1-up and M2a-up gene sets, and in the ratio of M1 ssGSEA score to M2a ssGSEA score over time; student's t-tests. FIG. 18E presents graphical representations of M1 macrophage-specific genes at week 0, Welch's t-tests. FIG. 18F presents a graphical representation of M2a macrophage-specific genes at week 0, Welch's t-tests; **p<0.01, *p<0.05.

FIG. 19 presents a graphical representation of wound healing trends over time with relation to CCL1 expression.

FIG. 20A-FIG. 20B present graphical representations of results related to healing vs. non-healing wound types. FIG. 20A presents a graphical representation of results related to healing vs. non-healing wound types. FIG. 20B presents a graphical representation of results related to healing vs. non-healing wound types.

FIG. 21 presents graphical representations of gene expression in healing and non-healing wound types.

FIG. 22A-FIG. 22B present graphical representations of results related to gene expression in healing versus non-healing wound types. FIG. 22A presents a graphical representation of results related to gene expression in healing versus non-healing wound types. FIG. 22B presents a graphical representation of results related to gene expression in healing versus non-healing wound types.

FIG. 23A-FIG. 23G present results related to healing and non-healing wound types. FIG. 23A presents a volcano plot showing differences of non-healing compared to healing DFUs; log FC and p value of gene-wise negative binomial generalized linear model (glm). FIG. 23B presents graphical representations of genes identified as significant by glm with p<0.05 and log FC>|1.5| and verified with Welch's t-tests applied to normalized counts. FIG. 23C presents graphical representations of the significant difference in fold change of expression week 4 compared to week 0 and expression over time in individual subjects (dark line shows average).

FIG. 23D presents graphical representations of the significant difference in fold change of expression week 4 compared to week 0 and expression over time in individual subjects (dark line shows average). FIG. 23E presents graphical representations of simple linear regression of gene expression versus number of weeks to healing where slope was significantly non-zero at p<0.05. FIG. 23F presents graphical representations of changes in expression from week 0 to week 4 per patient; green line represents average expression. *p<0.05, **p<0.01, ***p<0.005.; * p<0.05. **p<0.01, Welch's t-test. FIG. 23G presents a chart showing how many subjects showed decreasing expression of the three genes shown in FIG. 23F over time.

FIG. 24A-FIG. 24D present results related to human gene expression and the microbiome. FIG. 24A presents a graphical representation of the relative abundance (left y-axis) of genera detected at >0.5% in healing and non-healing DFU microbiome. The Shannon index value (right y-axis) of each sample is indicated by a white circle. FIG. 24B presents genes with significant Pearson's correlation coefficient where r>|0.9| and p value <0.05 after Benjamini-Hochberg correction. FIG. 24C presents a diagram of the number of significantly positively correlated genes by gene set for each species or diversity measure. FIG. 24D presents a diagram of the number of significantly negatively correlated genes by gene set for each species or diversity measure.

FIG. 25A-FIG. 25F present results related to data normalization techniques. FIG. 25A presents a representation of hierarchical clustering at week 0. FIG. 25B presents a graphical representation of tSNE multidimensionality reduction. FIG. 25C presents graphical representations of significantly differentially expressed genes at week 0 (Fisher's combined p-value <0.01). FIG. 25D presents a representation of hierarchical clustering at week 4. FIG. 25E presents a graphical representation of tSNE multidimensionality reduction at week 4. FIG. 25F presents graphical representations of the top significantly differentially expressed genes at week 4 (Fisher's combined p-value <0.01).

FIG. 26A-FIG. 26D present graphical representations of results related to genes whose slopes over time significantly differed between healing and non-healing subjects over weeks 0 and 4 (FIG. 26A and FIG. 26B), and over weeks 0 and 12 (FIG. 26C and FIG. 26D,).

FIG. 27A-FIG. 27B present results related to healing and non-healing wounds. FIG. 27A presents a graphical representation of the relative abundance of genera detected at >0.5% in healing and non-healing DFU microbiome. FIG. 27B presents a correlation networks of genes or diversity metrics with human genes at week 0.

FIG. 28A-FIG. 28D present graphical representations of results related to the use of machine learning to predict gene ratios most predictive of healing outcome. FIG. 28A presents a graphical representation of the ratio of C3AR1 to CCL22 using week 0 samples, processed using z-score values. Positive values indicate higher expression of C3AR1 relative to CCL22 and negative values indicate lower expression of C3AR1 to CCL22. FIG. 28B presents a graphical representation of the combination of the ratio of C3AR1 to CCL22 with the ratio of RIMS2 to SIGLEC12 using logistic regression, which was identified as the most predictive combination of healing outcome using week 0 samples. The dotted line indicates the optimal location to separate between healing and non-healing groups, as identified by machine learning (logistic regression model shown). FIG. 28C presents a graphical representation of a receiver operator characteristic (ROC) curve of logistic regression model using C3AR1/CCL22 and RIMS2/SIGLEC12 for predicting healing outcome using all samples collected from 27 subjects between weeks 0 and 12. FIG. 28D presents a graphical representation of the prediction score for samples collected over time for healing and non-healing subjects. Values ranging from 0 to 1 correspond to likelihood of the sample belong to a healing subject, while values ranging from 0 to -1 correspond to likelihood of the sample belonging to a non-healing subject.

FIG. 29 presents coefficients of variation, determined from PLS-DA of DEGs, showing genes useful for discriminating between healing and non-healing DFUs, without discriminating between non-healing subtypes.

### DETAILED DESCRIPTION

In clinical care of DFUs, it is currently difficult to determine if a DFU is on a healing trajectory or not. As a result, clinicians have no objective way of knowing if they should continue a certain course of treatment or change treatments for their patients. Moreover, once they make the decision to discontinue a treatment and switch to a new one, they have no objective way of making the choice among the hundreds of different products available on the market. Some of these products have very different effects on the inflammatory response, which is critical for regulation of the wound healing process. For example, some products like amniotic membrane-derived materials are very anti-inflammatory, while other products like glutaraldehyde-crosslinked collagen matrices are very pro-inflammatory.

There is a need in the art for methods of determining whether a wound, such as a DFU, should be treated with anti-inflammatory or pro-inflammatory treatments, and for personalized medicine approaches to wound care. The present invention addresses this need.

Moreover, in the absence of disease, wound healing is a complex and dynamic process that occurs in four phases, each of which is regulated by macrophages with distinct phenotypes. In order for healing to occur, macrophages must transition from a pro-inflammatory (also called M1) to a pro-healing (also called M2) phenotype, although the extent of diversity of the M2 population in particular is not known.

Studies have investigated changes in immune-related processes in human chronic wounds using gene expression analyses of whole wound tissue because this technique is amenable to clinical sample collection, since samples can be easily collected into non-toxic and non-noxious buffers with minimal added time or effort. Some studies have used gene expression profiling to compare healing and non-healing DFUs. For instance, it was previously reported that the ratio of four M1 macrophage markers to three M2 macrophage markers in debrided wound tissue decreased over time for healing DFUs but increased for non-healing DFUs, although the genes were not specific to macrophages so conclusions about macrophage phenotype could not be drawn (Armstrong, D. G., et al., Int Wound J 4, 286-287 (2007); Nassiri, S., et al., The Journal of investigative dermatology 135, 1700-1703 (2015)). A small panel of macrophage-specific genes was analyzed and it was found that non-healing DFUs expressed relatively higher levels of M1 markers than healing DFUs (Ferraro, N. M., et al., Integr Biol (Camb) 9, 328-338 (2017)). However, these analyses did not clearly indicate which clinical action should be taken with a given result. For example, if the wound was found to be not healing, it remained unclear which treatment should be administered instead.

As such, the working examples of the instant disclosure in part compare changes in inflammation- and macrophage phenotype-related gene over time in human healing and non-healing DFUs and investigate the influence of the microbiome as a potential mediator, with results pointing to treatments most likely to heal that particular DFU. The identified differences between healing and non-healing DFUs are critical for understanding heterogeneity in the human response to treatment, with implications for the design of more personalized treatment strategies.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in molecular biology, immunology, animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, the terms “comprises,” “comprising,” “containing,” “having,” and the like can have the meaning ascribed to them in U.S. patent law and can mean “includes,” “including,” and the like.

As used herein, the term “healing” refers to the process by which a body repairs itself after injury. The healing process can include several stages such as hemostasis (blood clotting), inflammation, proliferation (growth of new tissue), and maturation (remodeling). Embodiments of the invention can be used to make predictions regarding whether the wound will progress through all or the rest of the healing process without the need for enhanced techniques or can be utilized to make predictions regarding whether wound will progress to a particular stage of healing (e.g., proliferation) without the need for enhanced techniques.

As used herein, the term “high-throughput screening” refers to a screening method or system that allows analysis of a large number of samples by analyzing the presence, absence, relative levels, or response in one or more measurements including, but not limited to, nucleic acid makeup, gene expression, protein levels, functional activity, response to a stimulus, etc.

The terms “induce,” and “induction” refer to the promoting a change in macrophage phenotype from one macrophage phenotype to another macrophage phenotype.

The terms “isolated,” “purified,” or “biologically pure” refer to material that is free to varying degrees from components which normally accompany it as found in its native state. “Isolated” denotes a degree of separation from original source or surroundings. “Purified” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified. “Purified” can also refer to a molecule separated after a bioconjugation technique from those molecules that were not efficiently conjugated.

The phrase “macrophage conversion” as used herein refers to the sequential change in macrophage phenotype, e.g., a macrophage transitioning from pro-inflammatory (M1) to pro-healing, including multiple M2 subtypes (e.g. M2a, M2c, M2f, etc.).

The term “wound macrophage” as used herein refers to a hybrid population of macrophages in a wound including a spectrum of macrophage phenotypes and subtypes that include, but are not limited to, M0, M1, and M2 (including multiple subtypes) macrophages.

The term “M1 macrophage” as used herein refers to a macrophage phenotype. M1 macrophage are classically activated or exhibit an inflammatory macrophage phenotype. The M1 phenotype generally acts at early stages of wound healing.

The term “M2” broadly refers to macrophages that function in constructive processes particularly found at the later stages of successful wound healing and tissue repair. Major differences between M2a, M2b, M2c, and M2f macrophages exist in wound healing.

The term “M2a macrophage” as used herein refers to a macrophage subtype of pro-healing macrophages most commonly induced by stimulation with interleukin-4.

The term “M2c macrophage” as used herein refers to a macrophage subtype of pro-remodeling macrophages most commonly induced by stimulation with interleukin-10. M2c macrophages are involved in matrix and vascular remodeling and tissue repair.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

As used herein, the term “ratio” refers to a relationship between two numbers (e.g., scores, summations, and the like). Although, ratios can be expressed in a particular order (e.g., a to b or a:b), one of ordinary skill in the art will recognize that the underlying relationship between the numbers can be expressed in any order without losing the significance of the underlying relationship, although observation and correlation of trends based on the ration may need to be reversed. For example, if the values of a over time are (4, 10) and the values of b over time are (2, 4), the ratio a:b will equal (2, 2.5), while the ratio b:a will be (0.5, 0.4). Although the values of a and b are the same in both ratios, the ratios a:b and b:a are inverse and increase and decrease, respectively, over the time period.

As used herein, the term “initial medical encounter” encompasses one or more related interactions with one or more medical professionals. For example, if a subject visits her primary care provider's office regarding a wound, her interactions with a medical assistant, nurse, physician's assistant, and/or physician would constitute a single “medical encounter.” Likewise, a subject's interactions with a plurality of medical professionals during an emergency department visit would also constitute an “initial medical encounter.” The term “initial medical encounter” also encompasses the first interaction with a medical professional specializing in wound care. For example, a subject's first appointment with a wound clinic could be considered an “initial medical encounter.” The “initial medical encounter” can be the actual first or subsequent encounter with a medical professional. For example, a medical professional may not obtain a first sample until after the wound persists from a first appointment to a second appointment.

As used herein, the term “pro-inflammatory agent,” “pro-inflammatory treatment,” and the like, generally refer to agents and/or treatments that promote the M1 phenotype of macrophages and/or promotes inflammation and/or an inflammatory response. Examples of pro-inflammatory treatments, e.g., pro-inflammatory wound treatments, include, but are not limited to, glutaraldehyde-crosslinked materials, xenogeneic protein-derived materials, skin regeneration systems such as Integra® Dermal Regeneration Template, a biologic extracellular matrix such as Oasis® Wound Matrix (see Witherel et al. 2016 Wound Repair and Regeneration), biomaterials that promote the M1 phenotype in host macrophages through the release of agents (drugs, proteins, or nucleic acids), and M1 macrophage cell therapy. Examples of M1-promoting bioactive factors include activators of the inflammasome, NF-kappa-B, tumor necrosis factor, or interferon, or interleukin-1 pathways.

As used herein, the term “anti-inflammatory agent”, “anti-inflammatory treatment,” and the like, generally refer to agents and/or treatments that reduce inflammation, stimulate an “M2” response of macrophages (M2 promoting agent), and/or are regenerative agents. Examples of anti-inflammatory, regenerative, and/or stimulating an “M2” response of macrophages treatments include, but are not limited to, amniotic membrane or placental-derived tissues (see Witherel et al. 2017 Cellular and Molecular Bioengineering), bioengineered allogeneic cellular constructs such as Apligraf, some extracellular matrix-derived materials such as urinary bladder matrix (UBM), biomaterials that inhibit the M1 phenotype and/or promote the M2 phenotype in host macrophages through the release of bioactive factors (drugs, proteins, or nucleic acids), and M2 macrophage cell therapy. Examples of M1-inhibiting and/or M2-promoting bioactive factors include anti-inflammatory drugs, corticosteroids, Th2 cytokines like IL4, IL13, and IL10, and mesenchymal stem cells (MSCs).

As used herein, the term “sample” includes biological samples of materials such as organs, tissues, cells, fluids, and the like. In one embodiment, the sample can be obtained from a wound. In other embodiments, the sample can be obtained from inflamed tissue such as tissue afflicted with Inflammatory Bowel Syndrome, Crohn's disease, and the like. In still another embodiment, the tissue can be cancerous tissue (in which an increase in M1/M2 ratio would be desired for inhibition of tumor progression and a low or decreasing M1/M2 ratio would be indicative of tumor progression and metastasis). In still another embodiment, the sample can be obtained from an in vivo or in vitro testing platform such as a culture dish, a scaffold, an artificial organ, a laboratory animal, and the like.

As used herein, the term “treatment” or “treating” is defined as the application or administration of a therapeutic agent, i.e., a compound useful within the disclosure (alone or in combination with another pharmaceutical agent), to a patient/subject, or application or administration of a therapeutic agent to an isolated tissue or cell line from a patient/subject (e.g., for diagnosis or ex vivo applications), who has a disease or disorder and/or a symptom of a disease or disorder, with the purpose to cure, heal, alleviate, relieve, alter, remedy, ameliorate, improve or affect the disease or disorder and/or the symptoms of the disease or disorder. Such treatments may be specifically tailored or modified, based on knowledge obtained from the field of pharmacogenomics.

As used herein, the term “wound” includes injuries in which the skin (particularly, the dermis) is torn, cut, or punctured. Examples of types of wounds that can be assessed using embodiments of the invention described herein include external wounds, internal wounds, clean wounds (e.g., those made in the course of a medical procedure such as surgery), contaminated wounds, infected wounds, colonized wounds, incisions, lacerations, abrasions, avulsions, puncture wounds, penetration wounds, gunshot wounds, and the like. Specific wound examples include diabetic ulcers, pressure ulcers (also known as decubitus ulcers or bedsores), chronic venous ulcers, burns, and medical implant insertion points. Embodiments of the invention are particularly useful in identifying nonhealing wounds that are prevalent in diabetic and/or elderly subjects.

Methods of Treating and/or Ameliorating Wounds

Using diabetic foot ulcer as a non-limiting example, the present study discovered that molecular signatures measured from wound tissue can be used to determine the type of treatment a patient should be given. In some instances, non-healing wounds are classified as either of two subtypes: non-healing subtype 1 (hypo-inflammatory) and non-healing subtype 2 (hyper-inflammatory). As discussed herein, the present disclosure including the working examples present analysis that identified these two distinct subtypes of non-healing wounds, and that, based on the identified subtype, either a pro-inflammatory or an anti-inflammatory treatment should be administered. For instance, if the tissue exhibits a hypo-inflammatory molecular signature resembling “non-healing subtype 1,” they should be treated with more pro-inflammatory treatment options. Anti-inflammatory treatments should be contraindicated. If the tissue exhibits a hyper-inflammatory molecular signature resembling “non-healing subtype 2,” then the patient should be treated with more anti-inflammatory treatments, or those that promote a more regenerative “M2-like” phenotype of macrophages.

Examples of pro-inflammatory wound treatments include, but are not limited to, glutaraldehyde-crosslinked materials, xenogeneic protein-derived materials, skin regeneration systems such as Integra® Dermal Regeneration Template, a biologic extracellular matrix such as Oasis® Wound Matrix (see Witherel et al. 2016 Wound Repair and Regeneration), biomaterials that promote the M1 phenotype in host macrophages through the release of agents (drugs, proteins, or nucleic acids), and M1 macrophage cell therapy. Examples of M1-promoting bioactive factors include activators of the inflammasome, NF-kappa-B, tumor necrosis factor, or interferon, or interleukin-1 pathways.

Examples of anti-inflammatory, regenerative, and/or stimulating an “M2” response of macrophages treatments include, but are not limited to, amniotic membrane or placental-derived tissues (see Witherel et al. 2017 Cellular and Molecular Bioengineering), bioengineered allogeneic cellular constructs such as Apligraf, some extracellular matrix-derived materials such as urinary bladder matrix (UBM), biomaterials that inhibit the M1 phenotype and/or promote the M2 phenotype in host macrophages through the release of bioactive factors (drugs, proteins, or nucleic acids), and M2 macrophage cell therapy. Examples of M1-inhibiting and/or M2-promoting bioactive factors include anti-inflammatory drugs, corticosteroids, Th2 cytokines like IL4, IL13, and IL10, and mesenchymal stem cells (MSCs).

Accordingly, in some aspects, the instant specification is directed to a method of treating and/or ameliorating a wound (e.g., a non-healing wound) in a subject in need thereof. The method comprises measuring the levels of expression of a first panel of inflammation-related genes from a sample from the wound, and measuring the levels of expression of a second panel of inflammation-related genes from the sample. When at least one gene from the first panel (or a composite score of genes from the first panel or a ratio of genes compared to the second panel) is expressed at a lower level than a reference sample, a pro-inflammatory agent is administered to the subject. When at least one gene from the second panel or a composite score of genes from the second panel or a ratio of genes compared to the first panel is expressed at a higher level than a reference sample, an anti-inflammatory or M2-promoting agent is administered to the subject. Thus, the wound is treated and/or ameliorated in the subject.

In certain embodiments, the method of treating and/or ameliorating a wound (e.g., a non-healing wound) comprises measuring expression of a first and second panel of genes from a sample from the wound. In some aspects, the first panel and/or the second panel comprises RIMS2, CXCL11, EBI3, ST8SIA6, IFNG, IL6, IL3RA, FCGR2B, TLR2, SPP1, IL15, TNFRSF1B. In some aspects, the first panel and/or the second panel comprises ADORA2A, ANKRD22, APOBEC3A, APOL1, ASPHD2, C1ORF61, CASP1, CCL1, CCL19, CCL5, CCL8, CCR7, CD38, CD80, CFB, CFH, CLEC4D, CLEC4E, CMPK2, CRISPLD2, CSF3, CXCL10, CXCL11, CXCL9, EBI3, EPHA2, FBXO2, GBP1, GBP4, GBP5, GCH1, HAPLN3, HCAR3, HLA-DOA, HLA-DOB, HSH2D, IDO1, IDO2, IFI44L, IFITM1, IFITM3, IGFBP4, IL15, IL15RA, ILIB, IL27, IL32, IL3RA, IL6, IL8, IRF1, ISG15, ISG20, ITK, KRT7, LAG3, MN1, MT1M, NCF1, NCF1B, NEURL3, NNMT, NOD2, OASL, PDE4B, PRSS8, PTGES, PTGS2, RIMS2, RSAD2, SERPINB7, SERPING1, TNFAIP6, TNIP3, UBD, VEGFA, XAF1, and ZBP1. When at least one gene from the first panel or a composite score of genes from the first panel or a ratio compared to genes from the second panel is expressed at a lower level than a reference/control sample, a pro-inflammatory agent is administered to the subject. When at least one gene from the second panel or a composite score of genes from the second panel or a ratio compared to genes from the first panel is expressed at a higher level than a reference/control sample, an anti-inflammatory or M2 macrophage-promoting agent is administered to the subject. Thus, the wound is treated and/or ameliorated in the subject. This process may be repeated on a given wound until the wound is completely healed.

In some aspects, the ratio compared to genes from the first panel or the ratio compared to genes from the second panel is a ratio of C3AR1 to CCL22, a ratio of RIMS2 to SIGLEC12, or a combination of a ratio of C3AR1 to CCL22 and a ratio of RIMS2 to SIGLEC12. In some aspects, the ratio compared to genes from the first panel or the ratio compared to genes from the second panel is a ratio of C3AR1 to CCL22. In some aspects, the ratio compared to genes from the first panel or the ratio compared to genes from the second panel is a ratio of RIMS2 to SIGLEC12. In some aspects, the ratio compared to genes from the first panel or the ratio compared to genes from the second panel is a combination of a ratio of C3AR1 to CCL22 and a ratio of RIMS2 to SIGLEC12. In some aspects, the ratio comprises or is a ratio of an inflammatory gene to a reparative gene. In some aspects, the first panel comprises inflammatory genes including but not limited to ADORA2A, ANKRD22, APOBEC3A, APOL1, ASPHD2, C1ORF61, CASP1, CCL1, CCL19, CCL5, CCL8, CCR7, CD38, CD80, CFB, CFH, CLEC4D, CLEC4E, CMPK2, CRISPLD2, CSF3, CXCL10, CXCL11, CXCL9, EBI3, EPHA2, FBXO2, GBP1, GBP4, GBP5, GCH1, HAPLN3, HCAR3, HLA-DOA, HLA-DOB, HSH2D, IDO1, IDO2, IFI44L, IFITM1, IFITM3, IGFBP4, IL15, IL15RA, IL1B, IL27, IL32, IL3RA, IL6, IL8, IRF1, ISG15, ISG20, ITK, KRT7, LAG3, MN1, MTIM, NCF1, NCF1B, NEURL3, NNMT, NOD2, OASL, PDE4B, PRSS8, PTGES, PTGS2, RIMS2, RSAD2, SERPINB7, SERPING1, TNFAIP6, TNIP3, UBD, VEGFA, XAF1, and ZBP1. In some aspects, the second panel comprises reparative genes including but not limited to ABCG2, ALDH1A2, ALOX15, ATOH8, CABLES1, CACNA1G, CACNB4, CCL22, CCL26, CCL28, CCDC85C, CD1C, CDH1, CH25H, CHDH, CLEC4G, COL5A3, CR2, CRB2, DACT1, DNASE1L3, DUOX1, DUOXA1, EHF, ENHO, FABP4, FAM110B, FCGR2B, FOXQ1, GCNT3, IL17RB, IL21R, LIMA1, LRRC4, MAOA, MEST, MORC4, MRC1, MS4A6E, MUCL1, NEK10, NIPAL1, OLFML3, PALD1, PCSK1, PDGFB, PLCB1, PLEKHA6, RAMP1, S100A1, SEMA3G, SIGLEC12, SLC25A48, SNAI3, ST8SIA6, SYT17, TAL1, TGM2, TIMP3, TNFRSF11A, TSPAN7, VTN, WDR66, WNT5B. In some aspects, the ratio is a ratio of a gene from the first panel of inflammatory genes to a gene from the second panel of reparative genes. In some aspects, the ratio is combined with 1, 2, 3, 4, 5, 6, 7, 8, 9, 10, 20, 30, 40, 50, 75, or 100 other gene ratios. In some aspects, the ratio is a ratio taken at the week 0 time point. In some aspects, the ratio is a ratio taken a week 0, week 1, week 2, week 3, week 4, week 5, week 6, week 7, week 8, week 9, week 10, week 11, or week 12. In some aspects, the ratio is taken at a time point that is any time point.

Moreover, in one aspect, the present disclosure generally relates to a method of treating and/or ameliorating a non-healing wound in a subject in need thereof, the method comprising: measuring the levels of expression of a first panel of inflammation-related genes from a sample from the wound, and measuring the levels of expression of a second panel of M2 macrophage-related genes from the sample, wherein when the sample is classified by a previously-trained machine-learning algorithm as hypo-inflammatory, a pro-inflammatory agent is administered to the subject, and wherein when the sample is classified by a previously-trained machine-learning algorithm as hyper-inflammatory, an anti-inflammatory or M2-promoting agent is administered to the subject thus treating and/or ameliorating the wound in the subject. In some aspects, the second panel of M2 macrophage-related genes comprises ABCG2, ALDH1A2, ALOX15, ATOH8, CABLES1, CACNA1G, CACNB4, CCL22, CCL26, CCL28, CCDC85C, CD1C, CDH1, CH25H, CHDH, CLEC4G, COL5A3, CR2, CRB2, DACT1, DNASE1L3, DUOX1, DUOXA1, EHF, ENHO, FABP4, FAM110B, FCGR2B, FOXQ1, GCNT3, IL17RB, IL21R, LIMA1, LRRC4, MAOA, MEST, MORC4, MRC1, MS4A6E, MUCL1, NEK10, NIPAL1, OLFML3, PALD1, PCSK1, PDGFB, PLCB1, PLEKHA6, RAMP1, S100A1, SEMA3G, SIGLEC12, SLC25A48, SNAI3, ST8SIA6, SYT17, TAL1, TGM2, TIMP3, TNFRSF11A, TSPAN7, VTN, WDR66, WNT5B.

Furthermore, in one aspect, the present disclosure generally relates to a method of treating and/or ameliorating a non-healing wound in a subject in need thereof, the method comprising: measuring the levels of expression of a panel of M2 macrophage-associated genes in a subject, wherein when the sample is classified by a previously-trained machine-learning algorithm as hyper-inflammatory or having too little M2 macrophage-associated gene expression, an anti-inflammatory or M2-promoting agent is administered to the subject, thus treating and/or ameliorating the wound in the subject.

In certain embodiments, the method of treating and/or ameliorating a wound (e.g., a non-healing wound) comprises measuring the levels of expression of a panel of genes. In certain embodiments, the panel comprises 227 genes related to macrophage phenotype, crosstalk with microbes, and general wound healing, as described herein. The levels are calculated and a partial least squares-discriminant analysis (PLS-DA) algorithm is applied. In certain embodiments, genes with a variable importance in the projection (VIP) greater than 1 and an absolute value of coefficient of covariation of at least 0.5 are clustered and used to classify non-healing wounds into subtype I and II.

Also provided herein is a method of treating and/or ameliorating a non-healing wound in a subject in need thereof comprising measuring the levels of expression of a first panel of inflammation-related genes from a sample from the wound, and measuring the levels of expression of a second panel of inflammation-related genes from the sample. When the sample is classified by a previously-trained machine-learning algorithm as hypo-inflammatory, a pro-inflammatory agent is administered to the subject, and when the sample is classified by a previously-trained machine-learning algorithm as hyper-inflammatory, an anti-inflammatory or M2-promoting agent is administered to the subject. Thus, the wound is treated and/or ameliorated in the subject. In certain embodiments, the previously-trained machine-learning algorithm is a Partial Least-Squares Discriminant Analysis (PLS-DA) algorithm, support vector machine, or neural network.

In some embodiments, the reference sample is a sample from a healing wound. In some embodiments, the reference sample is a sample from a healing wound or unwounded tissue from the same subject. In some embodiments, the reference sample is a sample from a healing wound from a different subject. In some embodiments, the reference sample is a sample comprising healing wounds from multiple subjects. In some embodiments, the reference sample is a control sample. In some embodiments, the reference sample is a sample with known quantities of the genes/proteins being measured. In some embodiments, the reference sample is a standard curve. In some embodiments, the reference sample is macrophages prepared in vitro to exhibit typical M1 or M2 phenotypes.

In some embodiments, the wound is a nonhealing wound. In some embodiments, the wound is a chronic wound. In some embodiments, the wound has shown no significant progress toward healing (such as failed to achieve sufficient healing) in about 7 days, such as about 10 days, about 2 weeks, about 15 days, about 20 days, about 3 weeks, about 4 weeks, about 30 days, about 2 months, about 6 months, about 1 year, about 2 years, about 3 years, about 5 years or about 10 years. In some embodiments, the wound has shown no significant progress toward healing after standard care for the time period set forth above. In some embodiments, the wound is an infected wound such as an infected surgical wound or an infected traumatic wound; or an ulcer such as a diabetic ulcer (e.g., a diabetic foot ulcer), an arterial ulcer, a venous ulcer, a pressure ulcer, an ischemic ulcer, and the like.

In some embodiments, the sample is collected by swabbing the wound. In some embodiments, the sample is collected by debriding the wound and collecting the debrided tissue. Debridement is the medical removal of dead, damaged, or infected tissue of or associated with wounds. The removed tissues are used as samples according to the method herein in some embodiments.

In some aspects, mechanical debridement is used in which removal of a dressing from a wound that proceeded from moist to dry will non-selectively remove tissue adjacent to the dressing. This removed tissue can then be separated from the dressing (e.g., by scraping, rinsing, and the like) or total RNA can be directly isolated from the tissue while still attached to the dressing. Advantageously, harvesting of debrided tissue from removed dressings avoids the challenges associated with more invasive approaches and provides sufficient quantities of human wound tissues for quantitative analyses of the cellular content using tissue that would otherwise be discarded. The debrided wound tissue can be from one or more selected from the group consisting of: a diabetic ulcer, a pressure ulcer, a chronic venous ulcer, a burn, a wound caused by an autoimmune disease, a wound caused by Crohn's disease, a wound caused by atherosclerosis, a tumor, a medical implant insertion point, a surgical wound, a bone fracture, a tissue tear, and a tissue rupture.

In some aspects, surgical debridement can be performed using various surgical tools such as a scalpel, a laser, and the like. Advantageously, harvesting of debrided tissue avoids the challenges associated with more invasive approaches such as using punch biopsies while providing sufficient quantities of human wound tissues for quantitative analyses of the cellular content using tissue that would otherwise be discarded. Although relatively non-invasive procedures can be used, the samples used herein can also be obtained through invasive procedures such as punch biopsies, shave biopsies, incisional biopsies, excisional biopsies, curettage biopsies, saucerization biopsies, fine needle aspiration, and the like.

In some embodiments, the sample can be preserved and/or stabilized until further analysis can be performed. For example, the sample can be immersed in a stabilization reagent such as RNALATER® stabilization reagent available from QIAGEN of Venlo, Netherlands. In some embodiments, RNA can be extracted from the sample, for example by using a lysing agent such as the TRIZOL® Plus RNA Purification Kit available from Life Technologies of Grand Island, New York. In some embodiments, complementary DNA (cDNA) can be synthesized from the extracted RNA by using, for example, an APPLIED BIOSYSTEMS® High-Capacity cDNA Reverse Transcription Kit available from Life Technologies.

In some embodiments, expression of the genes is measured at the nucleotide level. In some embodiments, expression is measured at the protein level. Methods of quantifying nucleotides (DNA, RNA, or mRNA) or protein (or mRNAs or proteins of any genes) are well known in the art. Non-limiting examples of mRNA quantification methods include methods such as RT-PCR, quantitative PCR (qPCR), microarrays, northern blotting, RNA Sequencing (RNA-seq), and the like. None limiting examples of protein quantification methods include enzyme-linked immunoassay (ELISA), western blotting, and the like. In some embodiments, expression of one or more genes can be measured, for example, using quantitative polymerase chain reaction (qPCR), as described in K. L. Spiller et al., “The role of macrophage phenotype in vascularization of tissue engineering scaffolds,” 35 (15) Biomaterials 4477-88 (May 2014) (hereinafter “Spiller 2014”).

In some embodiments, gene expression is represented as an absolute quantity of a particular gene. In some embodiments, the absolute quantity of a gene is determined as a concentration of the gene, which can be estimated using, for example, total volume, total weight, total protein level, total RNA level, total DNA level, or total nucleic acid level as a reference.

In some embodiments, gene expression is represented as a ratio between a quantity of a particular gene and a quantity of a reference biomarker. In some embodiments, the reference biomarker is an mRNA or a protein.

In some embodiments, the subject is administering an additional treatment. Additional treatments can include, but are not limited to, debriding the wound, applying a compression wrapping, applying a compression stocking, applying dressings promoting a moist environment to the wound, applying a wound offloading device, applying a hyperbaric oxygen therapy, applying an antibiotic, administering an immunomodulation medication, or combinations thereof.

In some embodiments, the subject is a human.

### Microbiome

The wound microbiota is a potential factor that tunes immune cell phenotypes in chronic wounds. Microbes have been shown to regulate macrophage phenotype in animal studies and in vitro studies, but have not yet been linked to immune cell behavior in human chronic wounds. In the gut, the microbial fermentation product, butyrate, can facilitate M2 polarization, while promoting wound healing and attenuating pathogenic inflammation in colitis models. On the other hand, the production of deoxycholic acid, a microbial metabolite of bile, is stimulated by high fat diet, and promotes M1 macrophage polarization and pro-inflammatory responses. In human skin inflammation and wound healing, similar roles for the microbiota in immune cell behavior have not been investigated.

As such, in some aspects, the wound microbiota from a subject is analyzed, such as by nucleic acid sequencing, and the composition of the wound microbiota, that is, the types of bacteria present in the wound, is used to help guide wound treatment.

### WORKING EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Materials and Methods

### Experimental Design

Debrided tissue samples were collected from chronic DFUs from 27 subjects treated with the standard of care as well as paired samples 3-4 weeks later for a subset of these subjects (n=14) to analyze changes in human gene expression over time and microbial composition between healing and non-healing DFUs, defined based on whether wound closure was complete by 12 weeks from initial sample collection (FIG. 13). NanoString™ was used for multiplex gene expression analysis of a custom-curated panel of 227 genes related to macrophage phenotype, crosstalk with microbes, and general wound healing. The macrophage phenotype markers were selected based on a previous study in which RNA-seq was used to determine the top markers of macrophage phenotypes stimulated in vitro with interferon-gamma and lipopolysaccharide (M1), IL-4 and IL-13 (M2a), and IL-10 (M2c) (Lurier, E. B., et al. (2017) Immunobiology, 1-10). Therefore, the present study used these labels (M1, M2a, and M2c) to indicate why these genes were included on the panel, although it is acknowledged that it is not clear how well these in vitro-derived phenotypes map to macrophage phenotypes in vivo. NanoString was selected as the method of analysis as opposed to whole transcriptome analysis such as RNA-seq, to increase the number of patient samples that could be processed and to reduce the risks associated with RNA-seq, including biological and technical noise (Hansen, K. D. et al., Nature biotechnology 29, 572-573 (2011)), inconsistency in reporting methodology (Simoneau, J. et al., Briefings in Bioinformatics 22, 140-145, doi: 10.1093/bib/bbz124 (2019)), and processing constraints on low-quantity RNA samples (Ozsolak, F. & Milos, P. M. Nat Rev Genet 12, 87-98, doi: 10.1038/nrg2934 (2011)). The identified differences between healing and non-healing DFUs are critical for understanding heterogeneity in the human response to treatment, with implications for the design of more personalized treatment strategies. Additionally, culture-independent, high throughput 16S rRNA gene sequencing was utilized to characterize DFU microbiota, as culture-based methods are biased against anaerobes and other microbes that are difficult to grow in culture (Gardner, S. E., et al., Diabetes 62, 923, doi: 10.2337/db 12-0771 (2013)).

### Subject Recruitment and Sample Collection

Twenty-nine subjects (Table 1) were recruited over a 2-year period from the Drexel University Wound Healing Center after providing written consent and in compliance with the study protocol approved by the Drexel University Institutional Review Board. Inclusion criteria included being 18+ years of age with a diagnosis of type 2 diabetes and an ulcer that had been open for at least 8 weeks at the time of enrollment. Exclusion criteria included insufficient vascular perfusion (ankle brachial index <0.75) and those who presented with signs or symptoms of invasive or systemic infection such as cellulitis, abscess, or untreated osteomyelitis. Subjects could be enrolled after the cellulitis resolved and/or osteomyelitis was treated. Subjects were treated according to standard wound care procedures determined by the physician, including weekly or biweekly debridement with a sharp scalpel, offloading, topical antibiotics as needed, and moist wound dressings. Debrided DFU tissue samples were collected at every visit in which the physician deemed surgical debridement necessary until complete wound closure, amputation, death, or until the study ended (no earlier than 20 weeks since enrollment of the last subject). Debrided tissue samples were immediately collected into vials of RNALater as described below. The status of the wound was followed at subsequent clinical visits and samples were classified as healing or non-healing based on whether the wound was fully healed at 12 weeks from sample collection. Additionally, two subjects healed within 16-25 weeks from the initial sample collection, so the first samples collected from these subjects were counted as non-healing and then the sample collected exactly 12 weeks prior to healing was counted as a healing sample.

It was reasoned that the most useful samples to analyze would be initial sample collection as well as 3-4 weeks later because many treatment protocols calls for an initial 4-week period of the standard of care before switching to other more advanced treatments (Frykberg, R. G. & Banks, J. Adv Wound Care (New Rochelle) 4, 560-582, doi: 10.1089/wound.2015.0635 (2015)). Therefore, samples were collected at interim time points to optimize the RNA extraction protocol. Additionally, samples collected at the initial time point from 2 subjects yielded too little RNA for analysis, resulting in 27 subjects for analysis at the initial time point (n=12 healing and n=15 non-healing). For the subsequent time point, some samples yielded too little RNA for analysis, and some subjects were lost to follow-up, resulting in paired samples from n=6 healing DFUs and n=8 non-healing DFUs at the second time point 3-4 weeks later (FIG. 13).

Microbial analysis by 16S sequencing was conducted in parallel for the first 14 subjects who were enrolled as an initial pilot study to understand human-microbiome interactions. Of these, the initial sample collected from 5 subjects yielded microbial DNA that was of too low quality for sequencing and data analysis (details below), resulting in 9 subjects for microbial analysis (n=3 healing and n=6 non-healing) with paired human gene expression analysis.

TABLE 1
Subject characteristics.

| | Healing | Non-healing |
|---|---|---|
| | (n = 12) | (n = 15) |
| Average Age | 66.7 ± 13.25 | 62.2 ± 5.75 |
| Average BMI | 33.7 ± 5.04 | 36.8 ± 7.52 |
| % Female | 33.3 | 26.7 |
| % Current smoker | 33.3 | 40 |

### Debrided Tissue Sample Collection

Wound debridement was conducted with a #15 scalpel by the vascular surgeon director of the center after soaking the wound in 1% topical Xylocaine for at least 5 minutes prior to debridement. All subjects were debrided by the same surgeon. The entire wound was debrided down to bleeding granulation tissue using the #15 scalpel. Visible biofilm and slough were removed. Callous, if present, at the edge of the wound was excised. Tissue samples for the study were taken from the base of the wound after the visible bioburden and slough were removed. Hemostasis was obtained with the placement of a moist 4×4 gauze pad moistened with topical Xylocaine. Two samples were collected per wound. An initial sample was collected from the wound base and placed into RNALater for microbial analysis. Then, the remainder of the debrided tissue was collected into a second vial of RNALater for human gene expression analysis. Collected tissue samples were immediately placed in RNALater (Ambion, Carlsbad, CA) and stored at 4° C. overnight. They were then transferred to −80° C. until processing.

### Sequencing and Analysis of Microbial Composition

Bacterial DNA was extracted as described previously (Meisel, J. S. et al., J Invest Dermatol 136, 947-956, doi: 10.1016/j.jid.2016.01.016 (2016)) using the Invitrogen PureLink kit. PCR of the VIV3 hypervariable region was performed with barcoded primers (27F, 534R). Accuprime High Fidelity Taq polymerase was used for PCR cycling conditions: 94° C. for 3 min; 32 cycles of 94° C. for 45 sec, 50° C. for 60 sec, 72° C. for 90 sec; 72° C. for 10 min. PCR products were purified using the SequalPrep kit (Invitrogen), according to manufacturer's instructions, and pooled in equal amounts for sequencing. Barcoded amplicons were sequenced on the Illumina MiSeq platform using 300-bp paired end chemistry.

### RNA Extraction and NanoString

Tissue samples were thawed at room temperature, RNALater was removed, and total RNA was isolated using Trizol followed by purification with Qiagen RNeasy (Qiagen, Inc., CA, USA) according to manufacturer's protocol, as we have previously described (Nassiri, S., et al., The Journal of investigative dermatology 135, 1700-1703 (2015)). DNA was inactivated with DNAse I Amplification Grade (Invitrogen, Carsbad, CA, USA). NanoString gene expression analysis was run according to the manufacturer's recommended protocol using 100 ng per sample. Quantification and purity of RNA was assessed by spectrophotometric 260/280 ratio using a Nanodrop ND 2000 (ThermoFisher). 260/280 ratios ranged from 1.4-2.1 and they did not differ between healing and non-healing groups, so no samples were excluded from analysis, especially because NanoString has been shown to be reliable for gene expression analysis even in highly degraded tissue (Using the nCounter® Analysis System with FFPE Samples for Gene Expression Analysis. nCounter Gene Expression Tech Note (2012); Patel, P. G. et al., PLOS One 12, e0179732; Tsang, H. F. et al., Expert Rev Mol Diagn 17, 95-103). A custom code set of 227 genes was selected from the literature as being associated with distinct macrophage phenotypes (prepared in vitro), host-microbe communication, and others generally related to wound healing (Table 2). For macrophage phenotype markers, genes were further grouped based on whether they were previously found to be upregulated or downregulated with M1, M2a, or M2c polarization compared to unactivated controls (M0) using in vitro studies with defined chemical stimuli (lipopolysaccharide and interferon-gamma for M1, interleukin (IL)-4 and IL-13 for M2a, and IL 10 for M2c) (Ferraro, N. M., et al., Integr Biol (Camb) 9, 328-338 (2017); Lurier, E. B., et al., Immunobiology 222, 1-10 (2017)).

TABLE 2
Genes assessed using NanoString

| M1 | M2a | M2c | | | |
|---|---|---|---|---|---|
| (LPS + IFNg | (IL4 + IL13 | (IL10 | Wound | | |
| phenotype) | phenotype) | phenotype) | healing | Angiogenesis | Microbiome |
| ADORA2A | ABCG2 | CD163 | ABCC8 | ANGPT1 | C3AR1 |
| ANKRD22 | ALDH1A2 | CD300E | CASP1 | ANTP2 | C5AR1 |
| APOBEC3A | ALOX15 | F5 | CASP12 | CCL18 | CAMP |
| APOL1 | ATOH8 | FPR1 | CCNA1 | COL1A1 | CFD |
| ASPHD2 | CABLES1 | GXYLT2 | KDM5A | COL4A1 | DEFB1 |
| C1ORF61 | CACNA1G | KCNJ15 | KDM6B | CTGF | DEFB3 |
| CASP1 | CACNB4 | LIN7A | IL10 | FGF2 | DEFB4 |
| CCL1 | CCL22 | MERTK | NLRP3 | MMP7 | HC |
| CCL19 | CCL26 | MCTP2 | NUR77 | MMP9 | LYZ |
| CCL5 | CCL28 | PCOLCE2 | PI9 | NOTCH1 | MYD88 |
| CCL8 | CCDC85C | PLOD2 | SETD7 | PECAM1 | OR2AT4 |
| CCR7 | CD1C | SERPINA1 | TNFRSF1A | PF4 | REG3G |
| CD38 | CDH1 | SH3PXD2B | TNFRSF1B | SPP1 | S100A8 |
| CD80 | CH25H | TIMP1 | | TEK | S100A9 |
| CFB | CHDH | VCAN | | TGFB1 | SLPI |
| CFH | CLEC4G | | | TIE1 | TLR1 |
| CLEC4D | COL5A3 | | | TIE2 | TLR2 |
| CLEC4E | CR2 | | | | TLR3 |
| CMPK2 | CRB2 | | | | TLR4 |
| CRISPLD2 | DACT1 | | | | TLR5 |
| CSF3 | DNASE1L3 | | | | TLR6 |
| CXCl10 | DUOX1 | | | | TLR7 |
| CXCL11 | DUOXA1 | | | | TLR8 |
| CXCL9 | EHF | | | | TLR9 |
| EBI3 | ENHO | | | | |
| EPHA2 | FABP4 | | | | |
| FBXO2 | FAM110B | | | | |
| GBP1 | FCGR2B | | | | |
| GBP4 | FOXQ1 | | | | |
| GBP5 | GCNT3 | | | | |
| GCH1 | IL17RB | | | | |
| HAPLN3 | IL21R | | | | |
| HCAR3 | LIMA1 | | | | |
| HLA-DOA | LRRC4 | | | | |
| HLA-DOB | MAOA | | | | |
| HSH2D | MEST | | | | |
| IDO1 | MORC4 | | | | |
| IDO2 | MRC1 | | | | |
| IFI44L | MS4A6E | | | | |
| IFITM1 | MUCL1 | | | | |
| IFITM3 | NEK10 | | | | |
| IGFBP4 | NIPAL1 | | | | |
| IL15 | OLFML3 | | | | |
| IL15RA | PALD1 | | | | |
| IL1b | PCSK1 | | | | |
| IL27 | PDGFB | | | | |
| IL32 | PLCB1 | | | | |
| IL3RA | PLEKHA6 | | | | |
| IL6 | RAMP1 | | | | |
| IL8 | S100A1 | | | | |
| IRF1 | SEMA3G | | | | |
| ISG15 | SIGLEC12 | | | | |
| ISG20 | SLC25A48 | | | | |
| ITK | SNAI3 | | | | |
| KRT7 | ST8SIA6 | | | | |
| LAG3 | SYT17 | | | | |
| MN1 | TAL1 | | | | |
| MT1M | TGM2 | | | | |
| NCF1 | TIMP3 | | | | |
| NCF1B | TNFRSF11A | | | | |
| NEURL3 | TSPAN7 | | | | |
| NNMT | VTN | | | | |
| NOD2 | WDR66 | | | | |
| OASL | WNT5B | | | | |
| PDE4B | | | | | |
| PRSS8 | | | | | |
| PTGES | | | | | |
| PTGS2 | | | | | |
| RIMS2 | | | | | |
| RSAD2 | | | | | |
| SERPINB7 | | | | | |
| SERPING1 | | | | | |
| TNFAIP6 | | | | | |
| TNIP3 | | | | | |
| UBD | | | | | |
| VEGFA | | | | | |
| XAF1 | | | | | |
| ZBP1 | | | | | |

TABLE 3
Gene sets analyzed.

| Early stage genes | Late stage genes |
|---|---|
| ADORA2 | ABCG2 |
| APOBEC3A | CACNA1G |
| CCL1 | CACNB4 |
| CCL8 | CD1C |
| CCR7 | LRRC4 |
| CD38 | MEST |
| CD80 | MORC4 |
| CFB | OLFML3 |
| CLEC4E | TAL1 |
| CSF3 | TNFRSF11A |
| EBI3 | C1ORF61 |
| EPHA2 | IFI44L |
| GBP5 | MCTP2 |
| GCH1 | MN1 |
| HAPLN3 | |
| HCAR3 | |
| HLA-DOA | |
| HLA-DOB | |
| HSH2D | |
| IDO1 | |
| IDO2 | |
| IFITM1 | |
| IFITM3 | |
| IL15RA | |
| IL3RA | |
| IL6 | |
| IRF1 | |
| ISG20 | |
| ITK | |
| LAG3 | |
| MT1M | |
| NCF1 | |
| NCF1B | |
| NCF1C | |
| NNMT | |
| PDE4B | |
| PTGES | |
| PTGS2 | |
| RSAD2 | |
| TNIP3 | |
| UBD | |
| ZBP1 | |
| CCL22 | |
| CD163 | |
| CR2 | |
| DNASE1L3 | |
| FCGR2B | |
| FPR1 | |
| GCNT3 | |
| IL21R | |
| KCNJ15 | |
| LIN7A | |
| MS4A6E | |
| PALD1 | |
| PCSK1 | |
| PLOD2 | |
| SERPINA1 | |
| SLC25A48 | |
| SNAI3 | |
| TGM2 | |
| TIMP1 | |
| VTN | |

Note:
Genes in italics in the Early Stage gene set are also M1 macrophage phenotype markers, and genes in italics in the Late Stage gene set are also M2a macrophage phenotype markers.

### NanoString Data Processing

Raw counts from NanoString were normalized to internal positive and negative controls according to the manufacturer's recommendations. First, positive control normalization was performed by multiplying endogenous counts by their sample specific scaling factor, calculated using the geometric mean of all 6 positive controls divided by the average of geometric means for positive controls across all samples. Next, the background threshold method was used to account for noise. The average of the 8 negative controls was subtracted from all endogenous counts on per-sample basis. These steps were performed for enrollment and week 4 data sets separately. Samples were excluded from analysis if more than 50% of genes were not expressed above background levels.

Significantly differentially expressed genes (DEGs) were identified between healing (H) and non-healing (NH) groups using Welch's t-test and p-values <0.05 and log 2 fold change greater than 1.5. DEGs were plotted as heatmaps using the ComplexHeatmap package in R and bar plots were created in GraphPad Prism. For macrophage-specific analyses, the 12 genes were plotted individually. Lastly, gene set enrichment scores were calculated for all gene sets (Table 3) using the ssGSEA function of the GSVA package. Student's t-test was used to determine significance between groups at a p-value of 0.05. To evaluate changes over time, the fold change values of individual genes or ssGSEA scores at week 4 compared to week 0 were calculated as well as the ratio of these changes. Student's t-tests were used to establish significance between healing and non-healing DFU samples. P values were not adjusted for multiple comparisons because a small number of genes was investigated and a log 2 FC of at least 1.5 was used for significance.

### Partial Least Squares Discriminant Analysis (PLS-DA)

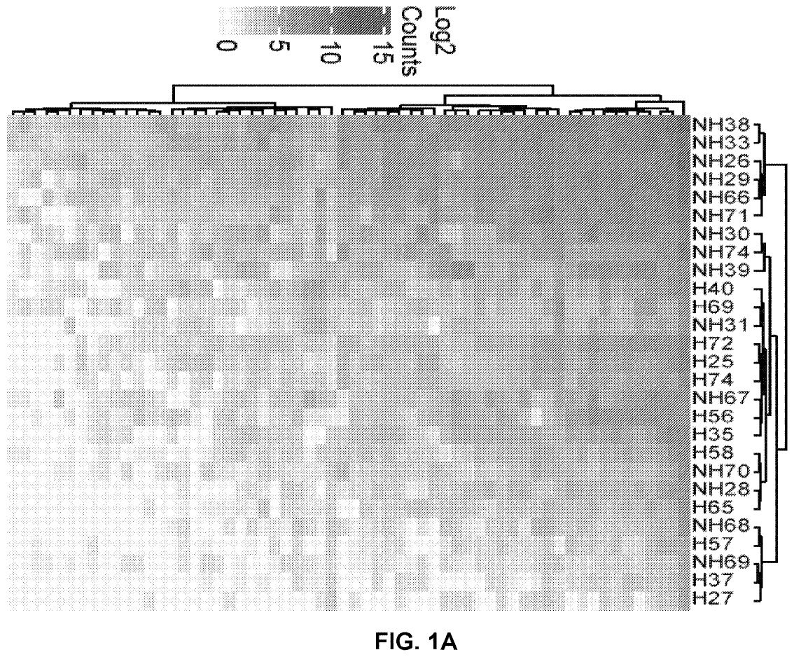

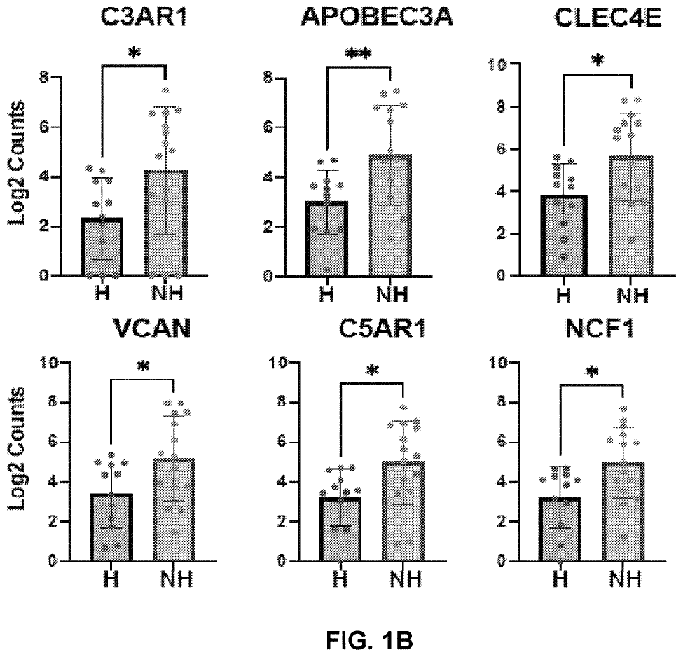

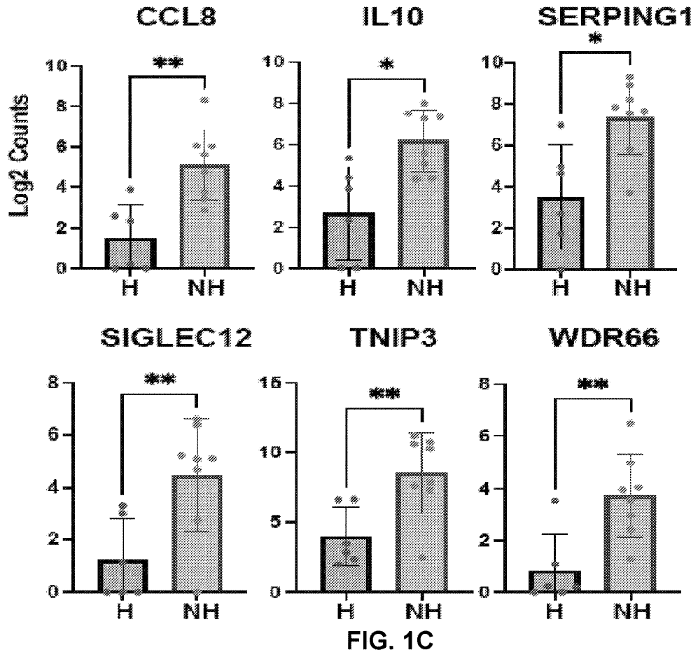

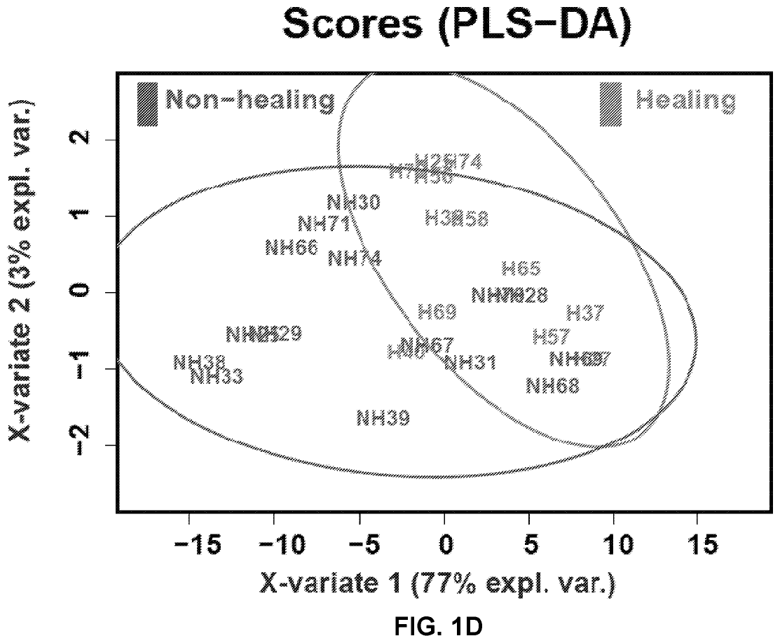

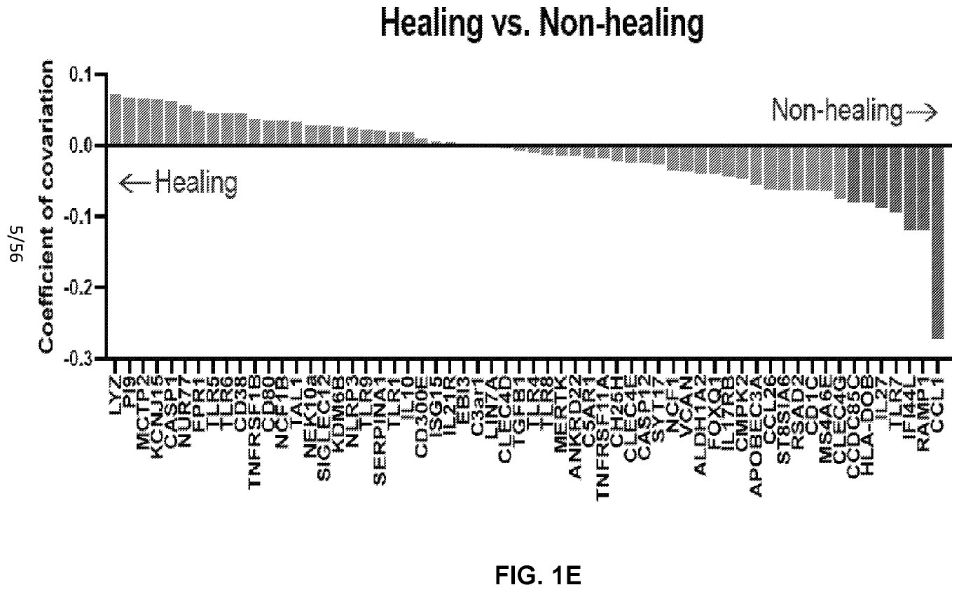

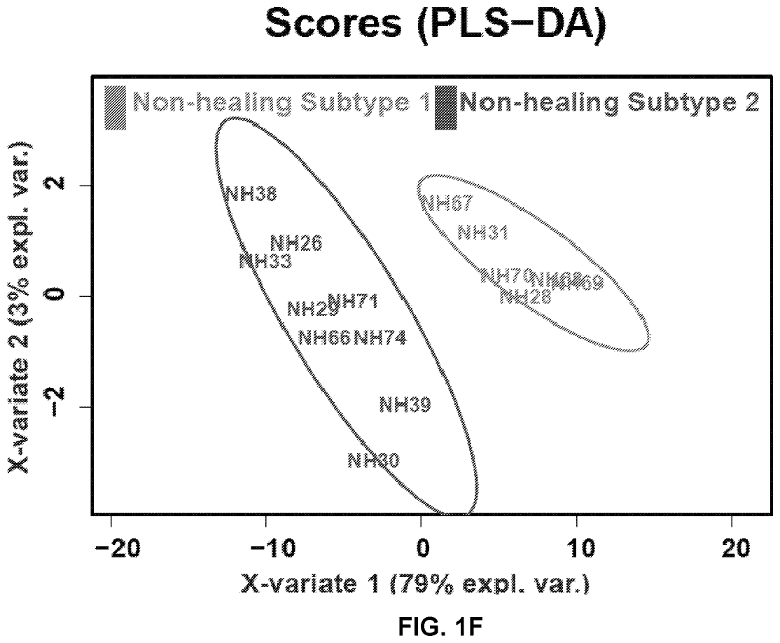

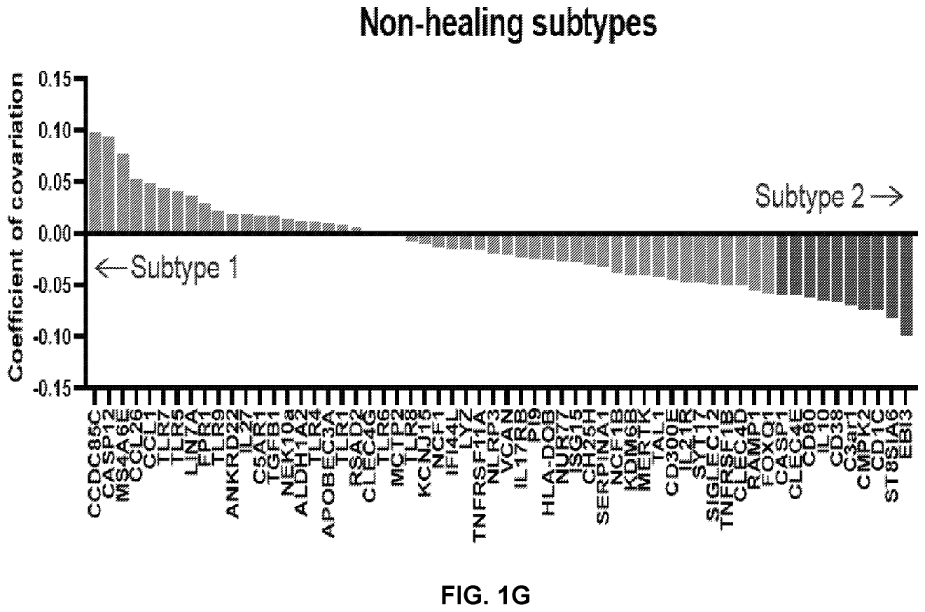

Partial least squares discriminant analysis (PLS-DA) was used as a multivariate approach to identify a molecular signature between healing and non-healing wounds as well as subtypes of non-healing wounds. A PLS-DA model was applied using the ropls package in R. Genes considered key in distinguishing outcomes were those with a variable importance in the projection (VIP) greater than 1 and an absolute value of coefficient of covariation of at least 0.5. Non-healing wounds were separated into subtype 1 and 2 based upon the hierarchical clustering of the DEGs between healing and non-healing DFUs as shown in FIG. 1F. Ellipses were drawn to indicate Mahalanobis distance based on default parameters. The top 12 genes most contributing to separation between non-healing subtypes were plotted and significant differential expression was determined via one-way ANOVA followed by Tukey's multiple comparisons tests. Welch's t-tests were used to determine genes differentially expressed between healing and non-healing subtype 1 groups.

### Upper Quartile Normalization for Analyses of Cellular Behavior

To account for differences in the quantity of immune cells between groups, data were then normalized to the upper quartile (UQ) using the EDASeq package in R. This method is typically applied to bulk RNA-seq data, but has been shown effective for reducing unwanted variation within NanoString data (Bhattacharya, A. et al. Briefings in bioinformatics 22, bbaa163, doi: 10.1093/bib/bbaa163 (2021)). The edgeR library was used for gene-wise negative binomial generalized linear modeling (glm) to estimate statistical significance between groups. Log 2 fold change values of healing vs. non-healing DFUs and −log 10 p-values were used to create a volcano plot with ggplot2. Genes were identified as statistically significant between groups if both the absolute value of log 2 fold change was greater than 1.5 and the p-value was less than 0.05. Significant DEGs were visualized with a heatmap using the ComplexHeatmap package. Identified genes were plotted in Prism using the normalized counts, not fitted, and confirmed to be statistically significant between groups using Welch's t-test and a significance level of p<0.05. Genes in which more than 50% of samples had no expression above negative controls were excluded. Fold change values of week 4 compared to week 0 were calculated for each gene for the subset of subjects with matched samples at both 0 week and 4 week time points. Welch's t-tests and criteria for significance were performed as previously described.

### Linear Regression Analysis

Linear regression analyses were performed for gene expression in the healing DFU samples as a function of number of weeks remaining until complete wound closure. The Im function within the stats package was used to calculate p-value and F-statistic. P-values were adjusted using the Benjamini-Hochberg (BH) correction. Genes with an adjusted p-value <0.05 and a ratio of F-statistic to number of samples greater than 0.5 were considered significant. Counts were then plotted in GraphPad Prism and the simple linear regression function was used to identify which genes had significantly non-zero slopes. Finally, these genes were analyzed on a per-subject level using matched samples for each subject using GraphPad Prism.

### Human-Microbe Correlation Analyses

Welch's t-tests were used to determine if there was statistical difference between diversity indexes or abundance of genera between healing and non-healing groups. Pearson's correlations were calculated for each gene to abundance of genera, Shannon index, phylogenetic diversity, and the number of observed species for the healing and non-healing samples separately. P-values were adjusted using the BH correction. Correlations with adjusted p-value <0.05 and |r|>0.9 were considered significant. Heatmaps were created using the ComplexHeatmap, RColorBrewer, and circlize packages in R. The non-healing dataset was clustered row-wise by Euclidean distance and single linkage. Pie charts representing the number of genes from each gene set were created in GraphPad Prism.

### Example 1: Multi-Gene Signatures, Wound Healing, and Wound Treatment

Gene expression signatures were determined herein that could be used to predict healing outcomes and determine which treatments (or class of treatments) would be most successful in terms of wound closure. Gene expression profiles were characterized in debrided wound tissue collected from chronic diabetic foot ulcers (DFUs) from 27 subjects using a custom-designed panel of 227 genes related to inflammation, macrophage phenotype, and wound healing. The DFUs were later determined to be healing or non-healing depending on whether their wound was fully closed at the 12-week time point following collection of the initial sample that was analyzed.

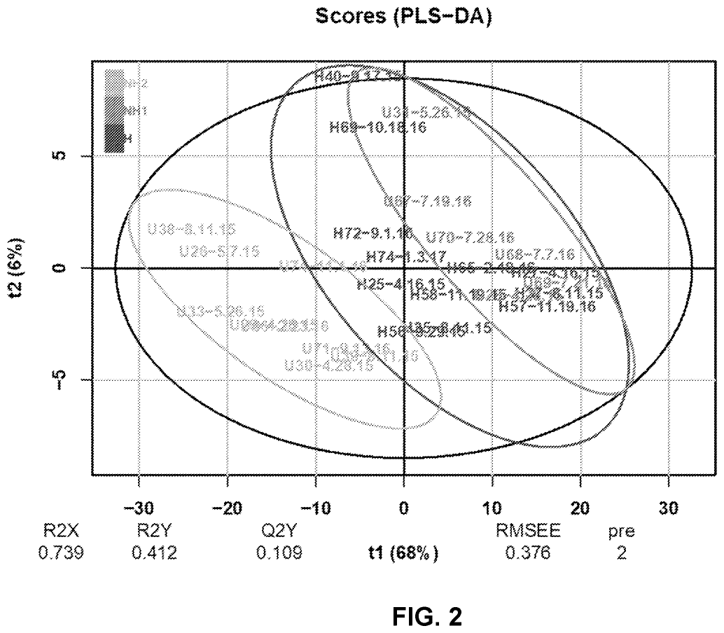

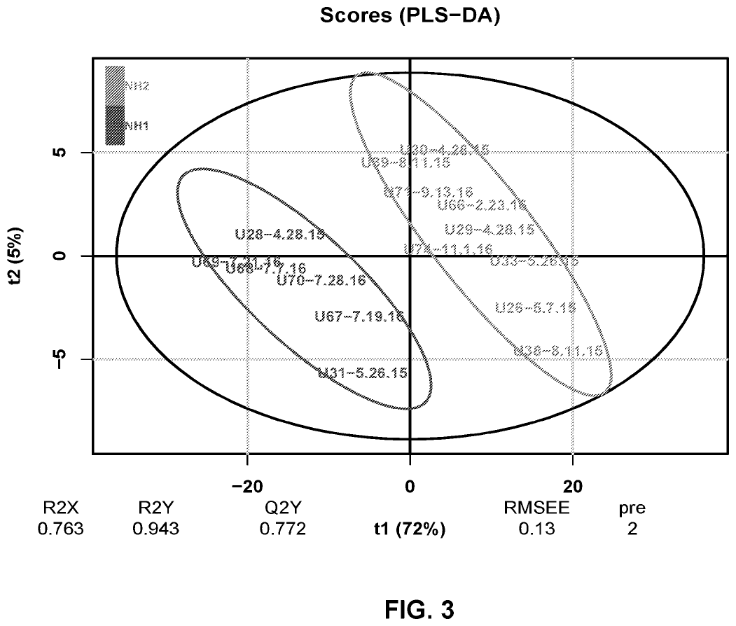

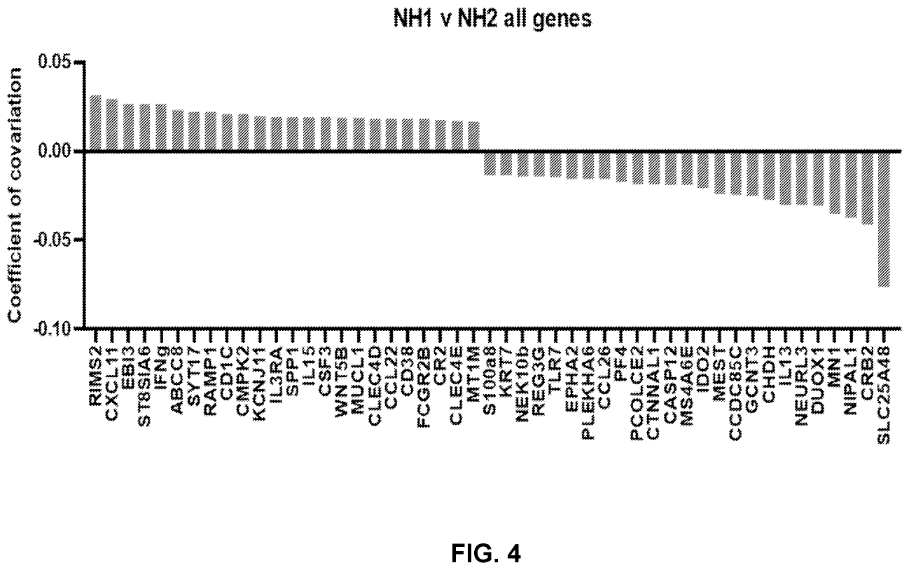

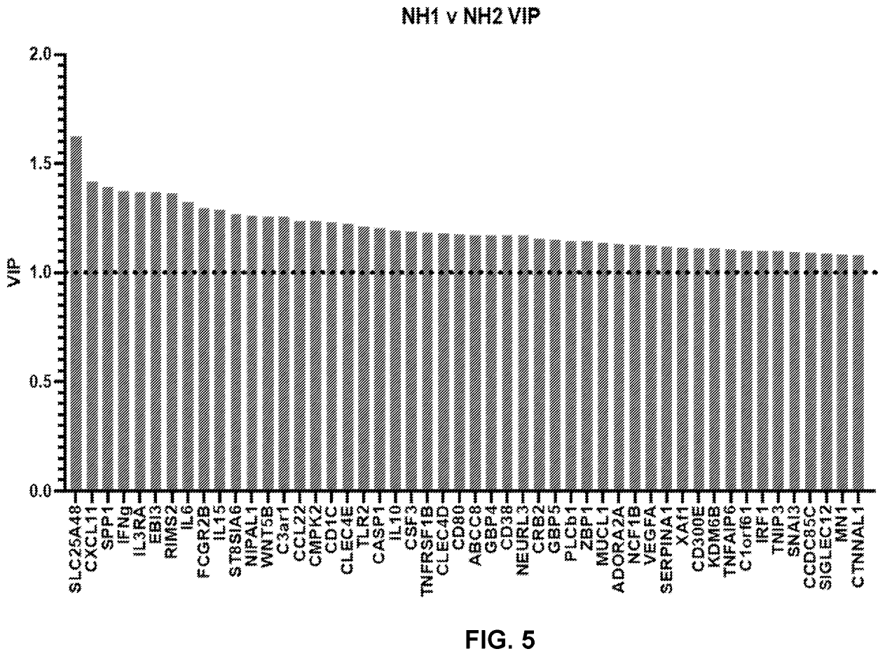

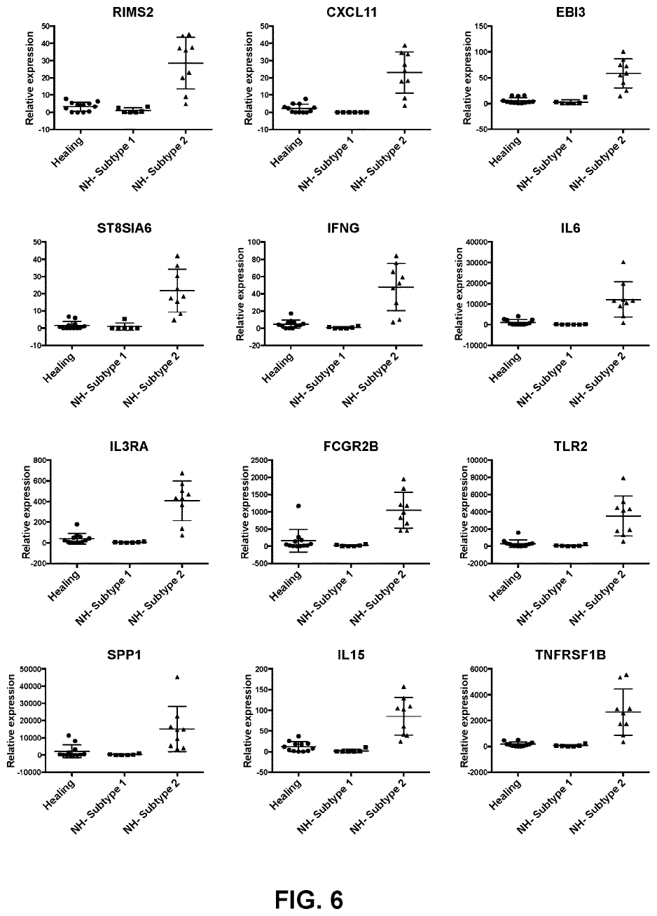

Hierarchical clustering of differentially expressed genes (DEGs) between healing (H) vs. non-healing (NH) DFUs showed that there are at least two distinct subtypes of non-healing DFUs: those that cluster together with the healing DFUs, and those that cluster separately from the healing DFUs (FIG. 1A, FIG. 1G). Partial least squares discriminant analysis (PLS-DA) was used to determine the differences between healing DFUs and the two non-healing subtypes (FIG. 2). This analysis showed clear separation between healing DFUs and the non-healing subtype 2, with less clear separation between healing DFUs and non-healing subtype 1. PLS-DA was repeated to determine the major differences between the two non-healing subtypes (FIG. 3). The coefficients of correlation (FIG. 4) and variables important for projection (FIG. 5) were calculated in order to identify genes that are useful for distinguishing between the two non-healing subtypes. The genes most associated with non-healing subtype 2 were largely associated with inflammation and the pro-inflammatory (M1) phenotype of macrophages, and they were expressed at higher levels in non-healing subtype 2 compared to both non-healing subtype 1 and to healing DFUs (FIG. 6), indicating that the non-healing subtype 2 DFUs are hyper-inflammatory. These results demonstrate that DFUs with the gene expression signature of non-healing subtype 2 should be treated with anti-inflammatory agents.

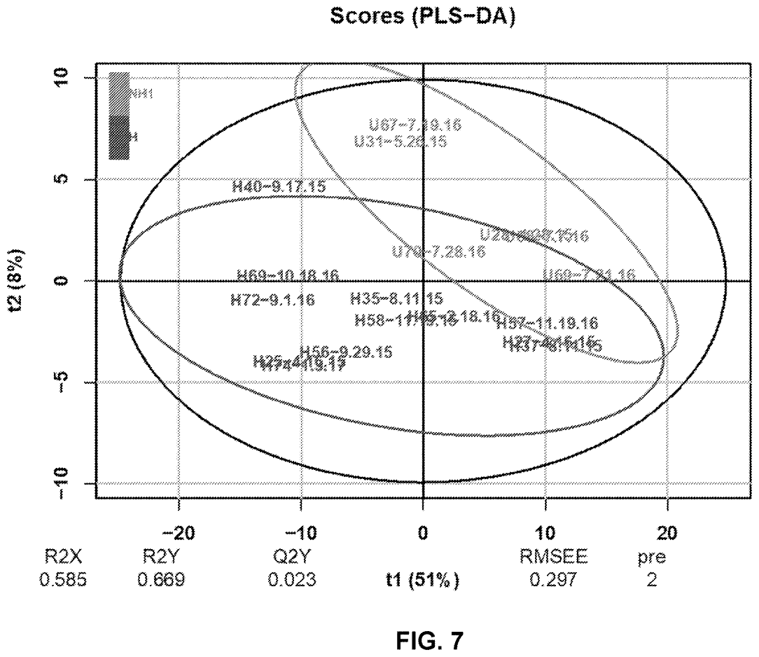

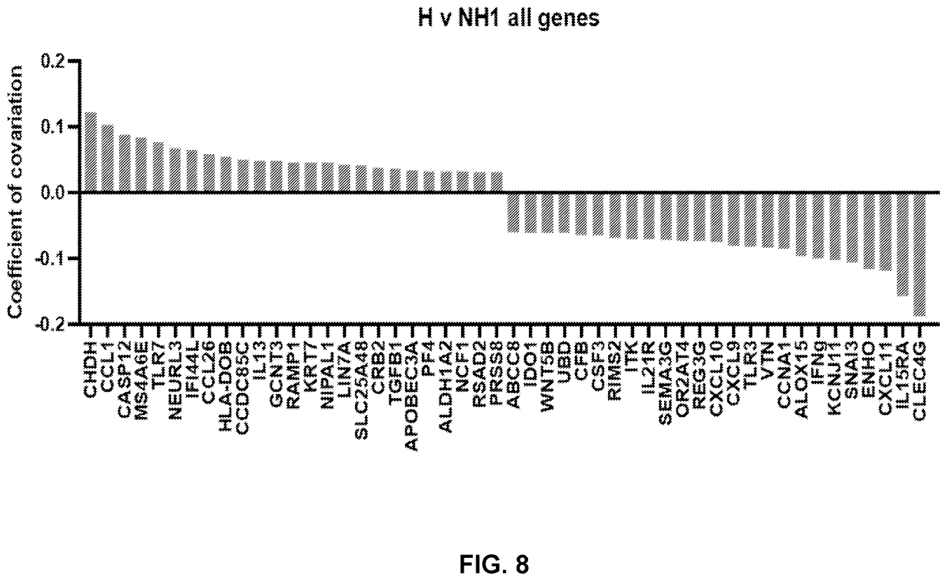

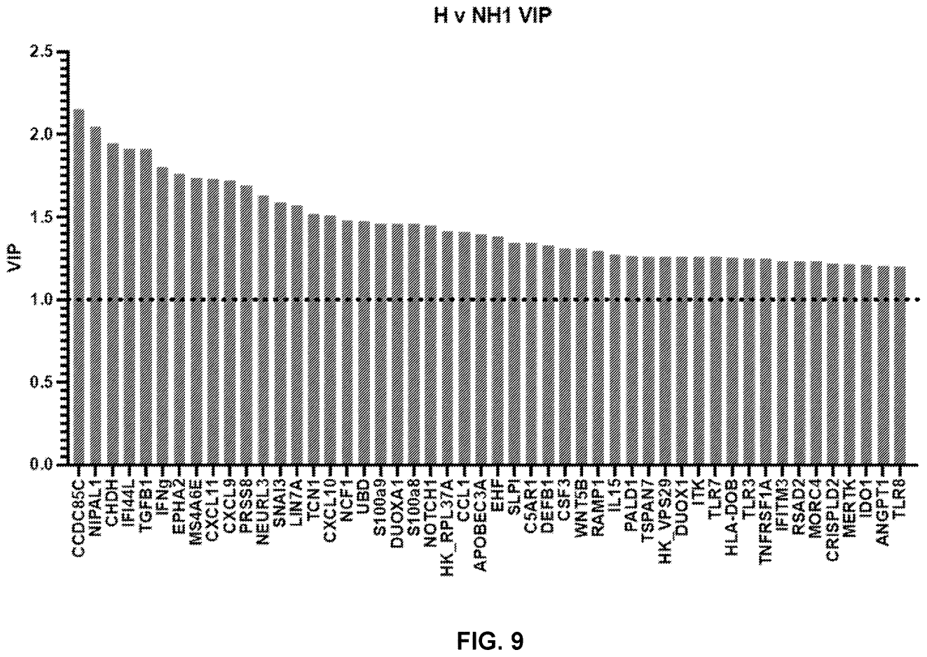

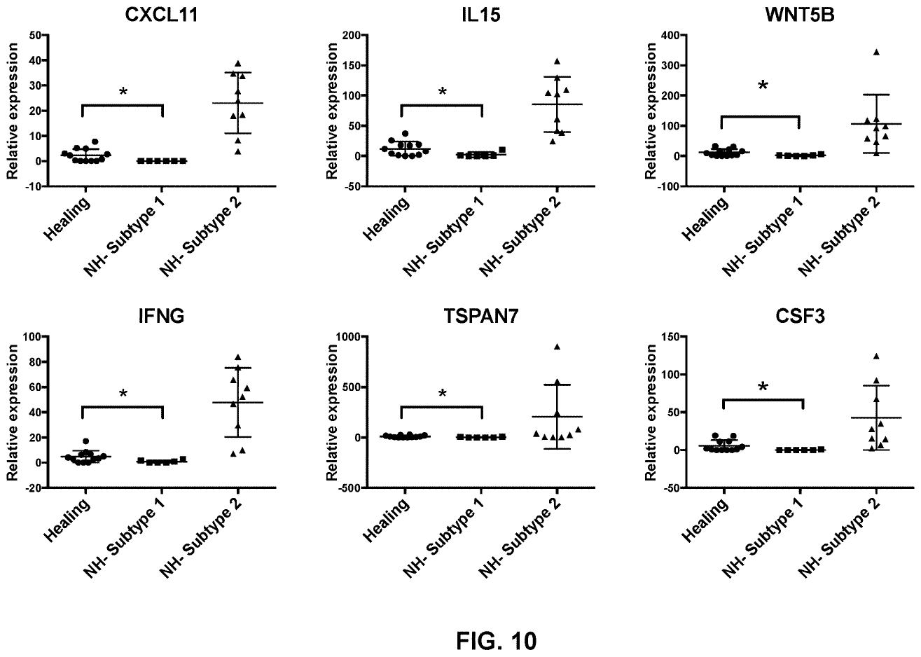

The next step was to distinguish between non-healing subtype 1 and healing wounds, which clustered together according to the DEG analysis. PLS-DA showed separation between non-healing subtype 1 and healing wounds (FIG. 7). Plotting coefficients of correlation (FIG. 8) and variables important for projection (FIG. 9) identified genes important for distinguishing between these two groups. This analysis also showed considerably lower expression of many genes associated with inflammation and the pro-inflammatory M1 phenotype of macrophages compared to healing DFUs and to non-healing subtype 2 DFUs (FIG. 10), indicating that the non-healing subtype 2 DFUs are hypo-inflammatory. These results show that DFUs with the gene expression signature of non-healing subtype 1 should be treated with pro-inflammatory agents.

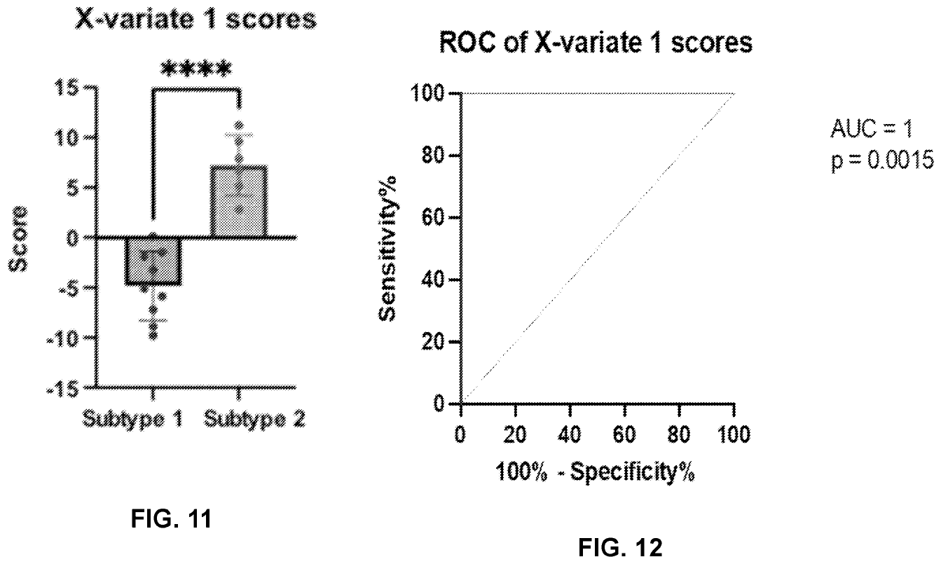

Finally, as an example of how to use a multi-gene expression signature to predict healing outcome and optimal treatment, PLS-DA covariates were used to predict non-healing subtypes (FIG. 11 and FIG. 12). The covariates showed no overlap between non-healing subtypes (FIG. 11) and a receiver operating characteristics (ROC) curve showed 100% sensitivity and specificity (FIG. 12). If this analysis predicts nonhealing subtype 2, then the patient is treated with an anti-inflammatory wound treatment or treatments. On the other hand, if the analysis predicts nonhealing subtype 1, then they should be further analyzed in order to differentiate them from a healing DFU. A composite of both x and y covariates from that PLS-DA analysis can be used to differentiate between nonhealing subtype 1 and healing DFUs. An ROC analysis of these scores also yielded 100% sensitivity and specificity. If this analysis predicts nonhealing subtype 1, then the patient is treated with a pro-inflammatory wound treatment or treatments. If the analysis predicts healing, then the patient should be continued to be treated as they were at the time of sample collection, or with the standard of care (moist wound dressings, debridement, offloading, etc.).

In summary, the present example describes multi-gene signatures to predict whether a wound will heal and whether they should be treated with more pro-inflammatory or more anti-inflammatory agents.

### Example 2: Differentially Expressed Genes (DEGs)

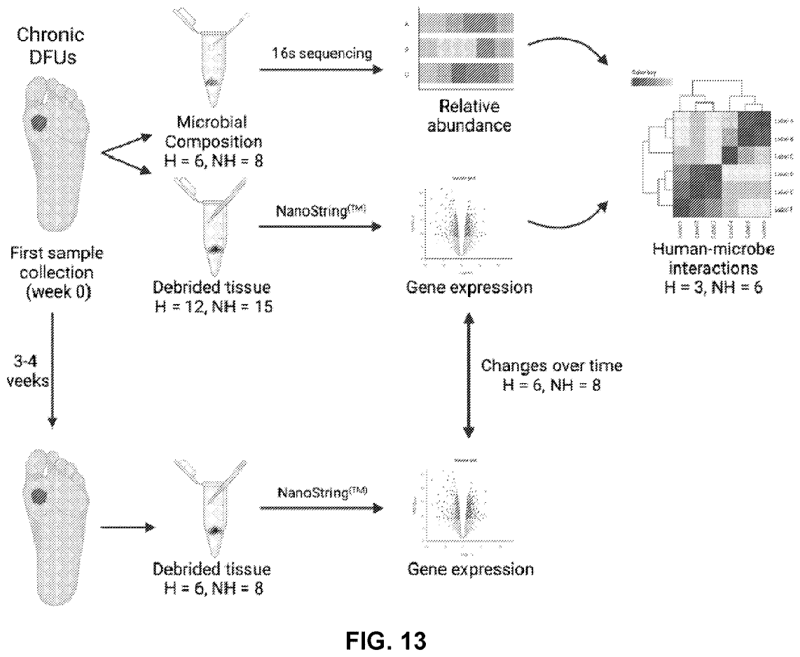

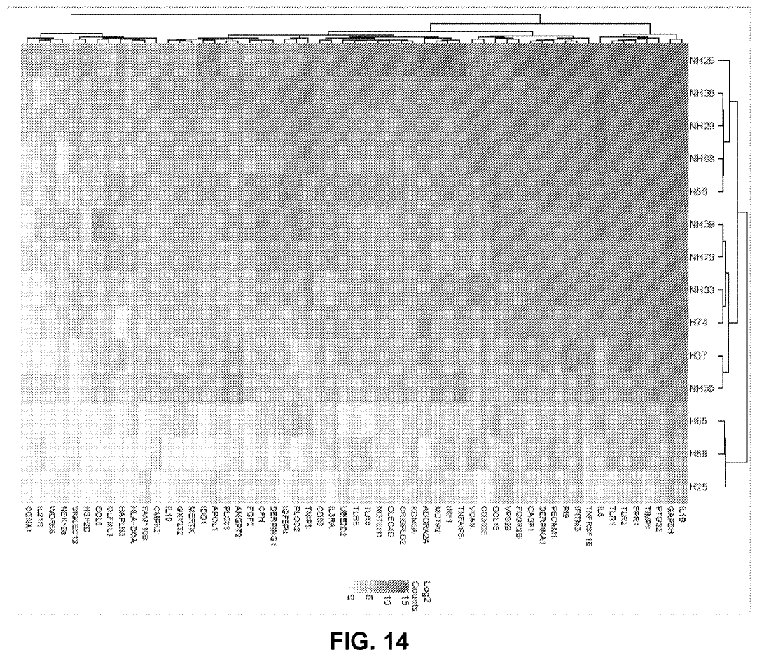

A custom-curated panel of 227 genes related to inflammation, macrophage phenotype, crosstalk with microbes, and wound healing was used more generally to characterize differences between DFUs that ultimately healed by 12 weeks (n=12) compared to those that remained open at that time point (n=15). There were 85 significant DEGs between these two groups at the first time point (Table 4) and 57 DEGs at week 4 (Table 5). All were significantly upregulated in non-healing DFUs compared to healing DFUs, with no significantly downregulated genes. Although a majority of the non-healing DFUs (9 of 15) exhibited higher expression of these inflammation-related genes compared to healing DFUs, 6 of the non-healing DFUs exhibited similar or lower levels, clustering together with healing DFUs (FIG. 1A). This pattern was also true at the 4 week time point, when the healing DFUs were all within 8 weeks of healing (FIG. 14).

TABLE 4
DEGs of positive and negative control
normalized data at enrollment

| Gene | P Value | logFC |
|---|---|---|
| EBI3 | 0.005 | 1.887 |
| SYT17 | 0.006 | 1.771 |
| ST8SIA6 | 0.006 | 2.141 |
| IL3RA | 0.007 | 1.830 |
| KDM6B | 0.007 | 1.931 |
| ITK | 0.008 | 1.890 |
| CASP1 | 0.008 | 1.710 |
| NOD2 | 0.008 | 1.848 |
| RIMS2 | 0.008 | 1.669 |
| CD80 | 0.008 | 1.935 |
| PLCb1 | 0.009 | 1.976 |
| NUR77 | 0.009 | 2.137 |
| CD1C | 0.009 | 2.132 |
| CXCL11 | 0.009 | 1.817 |
| TNFRSF1B | 0.010 | 2.273 |
| IFNg | 0.010 | 1.805 |
| VEGFA | 0.011 | 1.767 |
| NCF1B | 0.012 | 1.885 |
| C1orf61 | 0.012 | 1.524 |
| SERPINA1 | 0.013 | 2.324 |
| CD38 | 0.013 | 1.857 |
| TLR2 | 0.013 | 1.925 |
| APOBEC3A | 0.014 | 2.600 |
| MRC1 | 0.014 | 1.729 |
| TAL1 | 0.014 | 1.958 |
| C5AR1 | 0.016 | 2.415 |
| IL1B | 0.017 | 1.775 |
| IDO1 | 0.017 | 1.888 |
| CLEC4E | 0.017 | 2.455 |
| IL6 | 0.018 | 1.904 |
| ABCC8 | 0.018 | 1.573 |
| NLRP3 | 0.019 | 1.664 |
| KCNJ15 | 0.020 | 2.191 |
| PI9 | 0.020 | 2.115 |
| TLR4 | 0.020 | 2.416 |
| LIN7A | 0.021 | 2.180 |
| TLR6 | 0.021 | 2.310 |
| C3ar1 | 0.021 | 2.686 |
| MCTP2 | 0.021 | 1.885 |
| GCH1 | 0.022 | 1.609 |
| CMPK2 | 0.022 | 2.078 |
| TLR9 | 0.022 | 1.571 |
| VCAN | 0.023 | 2.389 |
| TNFRSF1A | 0.023 | 1.744 |
| PDE4B | 0.023 | 2.285 |
| TNFRSF11A | 0.023 | 1.953 |
| MYD88 | 0.024 | 1.698 |
| TLR7 | 0.026 | 2.206 |
| NOTCH1 | 0.026 | 1.554 |
| NCF1 | 0.027 | 2.208 |
| ADORA2A | 0.027 | 1.856 |
| CD163 | 0.027 | 2.163 |
| CCR7 | 0.027 | 1.981 |
| PCSK1 | 0.028 | 1.614 |
| XAf1 | 0.028 | 1.647 |
| TFRC | 0.028 | 1.656 |
| ANKRD22 | 0.029 | 1.915 |
| CLEC4G | 0.029 | 2.565 |
| MERTK | 0.029 | 1.980 |
| APOL1 | 0.030 | 1.607 |
| TNFAIP6 | 0.030 | 1.718 |
| F5 | 0.032 | 2.353 |
| CD300E | 0.032 | 2.628 |
| CFD | 0.033 | 2.189 |
| TGFB1 | 0.033 | 1.709 |
| SNAI3 | 0.034 | 1.829 |
| TIMP1 | 0.035 | 1.871 |
| CH25H | 0.035 | 1.736 |
| TLR1 | 0.037 | 2.242 |
| IL17RB | 0.038 | 1.928 |
| IL32 | 0.038 | 1.589 |
| IL21R | 0.038 | 2.449 |
| ISG15 | 0.038 | 2.387 |
| TLR8 | 0.038 | 2.412 |
| CCL26 | 0.039 | 1.537 |
| PLOD2 | 0.039 | 1.643 |
| WNT5B | 0.040 | 1.712 |
| CCL5 | 0.041 | 1.565 |
| IFI44L | 0.042 | 2.337 |
| SERPING1 | 0.042 | 1.673 |
| FPR1 | 0.044 | 2.284 |
| IFITM1 | 0.047 | 1.910 |
| SIGLEC12 | 0.048 | 2.026 |
| PECAM1 | 0.049 | 2.014 |
| TIMP3 | 0.049 | 1.592 |

TABLE 5
DEGs of raw data at week 4

| Gene | P value | logFC |
|---|---|---|
| CCL8 | 0.002 | 3.606 |
| WDR66 | 0.004 | 2.873 |
| TNIP3 | 0.005 | 4.542 |
| SIGLEC12 | 0.007 | 3.238 |
| IL10 | 0.010 | 3.532 |
| SERPING1 | 0.011 | 3.874 |
| IL21R | 0.014 | 2.224 |
| CRISPLD2 | 0.015 | 3.838 |
| OLFML3 | 0.015 | 3.132 |
| VCAN | 0.017 | 3.812 |
| TNFAIP6 | 0.019 | 3.824 |
| MERTK | 0.019 | 3.299 |
| MCTP2 | 0.020 | 3.142 |
| KDM5A | 0.020 | 2.778 |
| CFH | 0.020 | 3.361 |
| ANGPT2 | 0.023 | 3.179 |
| CMPK2 | 0.023 | 2.696 |
| FAM110B | 0.024 | 2.557 |
| FCGR2B | 0.025 | 3.697 |
| GXYLT2 | 0.027 | 2.834 |
| PLOD2 | 0.029 | 4.143 |
| IL6 | 0.030 | 4.703 |
| IRF1 | 0.030 | 3.310 |
| HSH2D | 0.030 | 2.620 |
| TNFRSF1B | 0.031 | 4.492 |
| NEK10a | 0.031 | 2.040 |
| HLA-DOA | 0.032 | 2.574 |
| CASP1 | 0.034 | 3.563 |
| HK_GAPDH | 0.035 | 3.129 |
| HK_VPS29 | 0.035 | 2.312 |
| NOTCH1 | 0.036 | 3.245 |
| IL3RA | 0.037 | 3.565 |
| IFITM3 | 0.037 | 3.175 |
| IL1B | 0.039 | 3.854 |
| IGFBP4 | 0.039 | 3.699 |
| CD300E | 0.040 | 3.763 |
| TLR2 | 0.040 | 3.688 |
| IDO1 | 0.041 | 3.462 |
| FPR1 | 0.041 | 3.573 |
| CCNA1 | 0.042 | 1.919 |
| FGF2 | 0.042 | 3.185 |
| PECAM1 | 0.042 | 3.074 |
| CCL18 | 0.043 | 3.505 |
| CLEC4D | 0.043 | 3.319 |
| APOL1 | 0.043 | 2.810 |
| SERPINA1 | 0.044 | 3.422 |
| TLR8 | 0.044 | 3.436 |
| PLCb1 | 0.045 | 2.776 |
| TLR6 | 0.045 | 2.883 |
| PTGS2 | 0.046 | 3.993 |
| HK_UBE2D2 | 0.046 | 3.356 |
| HAPLN3 | 0.048 | 3.012 |
| TIMP1 | 0.048 | 3.871 |
| ADORA2A | 0.048 | 3.603 |
| PI9 | 0.048 | 3.415 |
| CD80 | 0.048 | 3.183 |
| TLR1 | 0.049 | 3.793 |

Of the top 6 most differentially expressed genes between the groups (FIG. 1B), 3 were markers associated with the pro-inflammatory M1 macrophage phenotype (APOBEC3A, CLEC4E, and NCF1). C3AR1 and C5AR1 are involved in host-microbe communication, and VCAN was included as an M2c macrophage phenotype marker but is also involved in regulation of cell migration and extracellular matrix (ECM) assembly. At week 4, the top 6 most differentially expressed genes (FIG. 1C) were related to M1 (CCL8, TNIP3), M2a (SIGLEC12, WDR66), and the anti-inflammatory and M2c-promoting cytokine IL10, while SERPING1 is associated with downregulation of the M1 phenotype in macrophages. 28 genes were upregulated in non-healing DFUs at both time points (Table 6). Nine were M1 markers and 7 were M2c markers.

TABLE 6
Genes that were significantly upregulated by non-healers at both time points

| M1 | M2a | M2c | Angiogenesis | Bacteria | Wound Healing |
|---|---|---|---|---|---|
| APOL1 | IL21R | CD300E | NOTCH1 | TLR2 | CASP1 |
| CD80 | MCTP2 | FPR1 | VEGFA | TLR6 | PI9 |
| CMPK2 | PLCB1 | MERTK | | LR8 | |
| IDO1 | SIGLEC12 | PLOD2 | | | |
| IL1B | TNFS | SERPINA1 | | | |
| IL3RA | | TIMP1 | | | |
| IL6 | | VCAN | | | |
| SERPING1 | | | | | |
| NFAIP6 | | | | | |

### Example 3: Multi-Gene Molecular Signatures

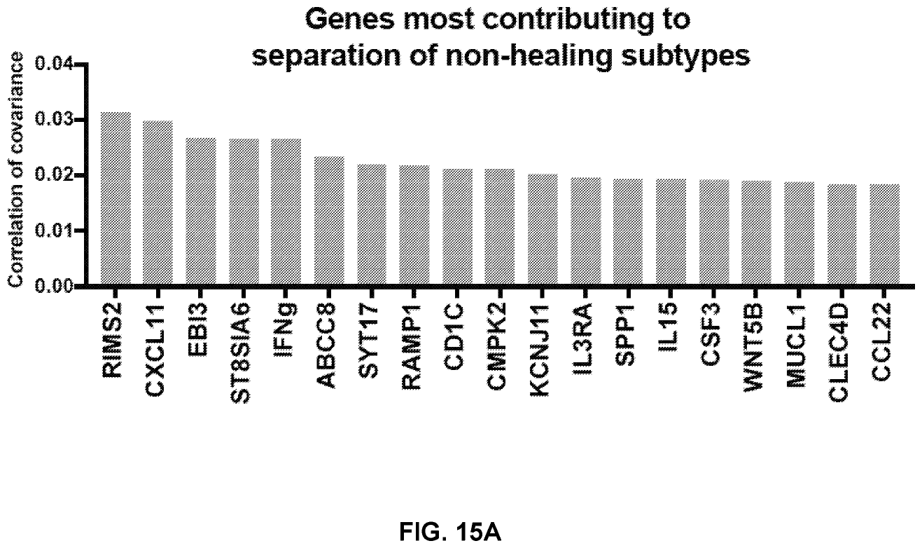

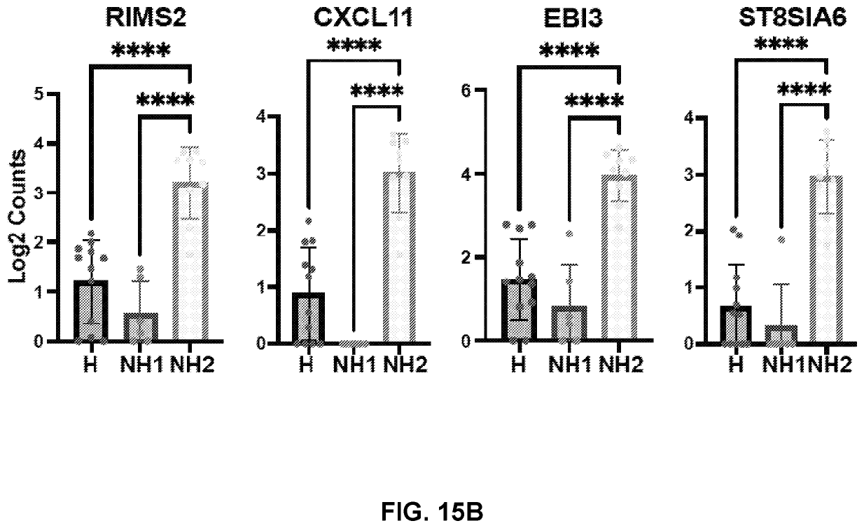

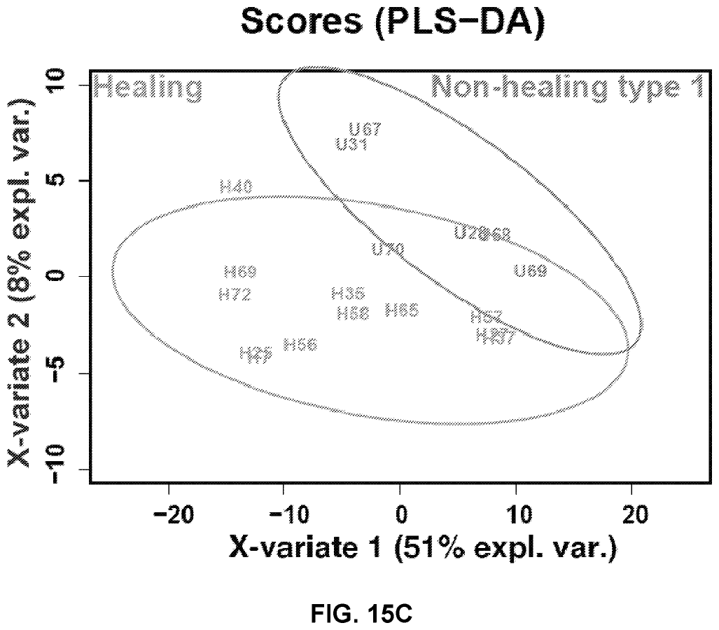

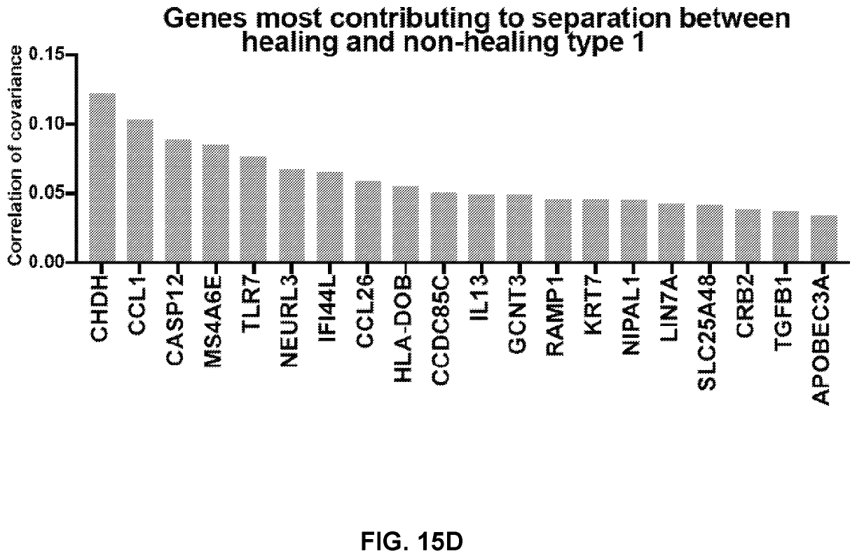

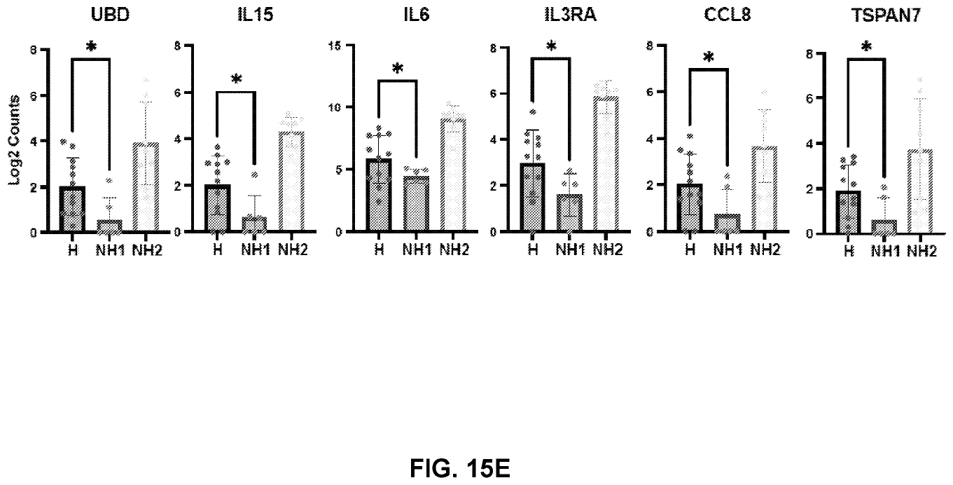

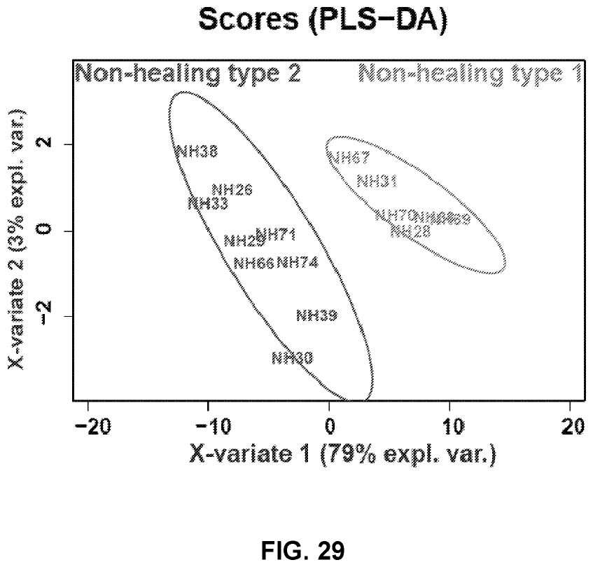

To further investigate the observed heterogeneity in the non-healing group, the dimensionality reduction method PLS-DA was used to interrogate the differences between the two sets of non-healing subtypes identified by hierarchical clustering (FIG. 1A). The subjects that clustered together with the healing DFUs were called subtype 1, while the subjects that clustered far from the healing DFUs were called subtype 2. The PLS-DA analysis showed clear separation between these subtypes (FIG. 1F; see also FIG. 29) and identified the genes that contributed most to this separation (FIG. 15A). The top 20 genes between non-healing subtypes were generally expressed at higher levels in subtype 2 compared to subtype 1, and they mostly related to the pro-inflammatory M1 phenotype and the early stages of healing (RIMS2, EBI3, CMPK, IL3RA, and CSF3) (FIG. 15A). The remainder of the genes were previously found to be upregulated by M2a in vitro yet downregulated in healthy wound healing (ST8SIA6, SYT17, RAMP1, CD1C, and MUCL1), strongly upregulated by M2a in vitro (CCL22), upregulated by M2c in vitro (KCNJ11), wound healing (ABCC8 and WNT5B), and angiogenesis (SPP1). The top 4 genes that contribute most to differences between the subtypes showed almost no overlap in expression between non-healing subtype 2 compared to non-healing subtype 1 or healing groups (FIG. 15B).

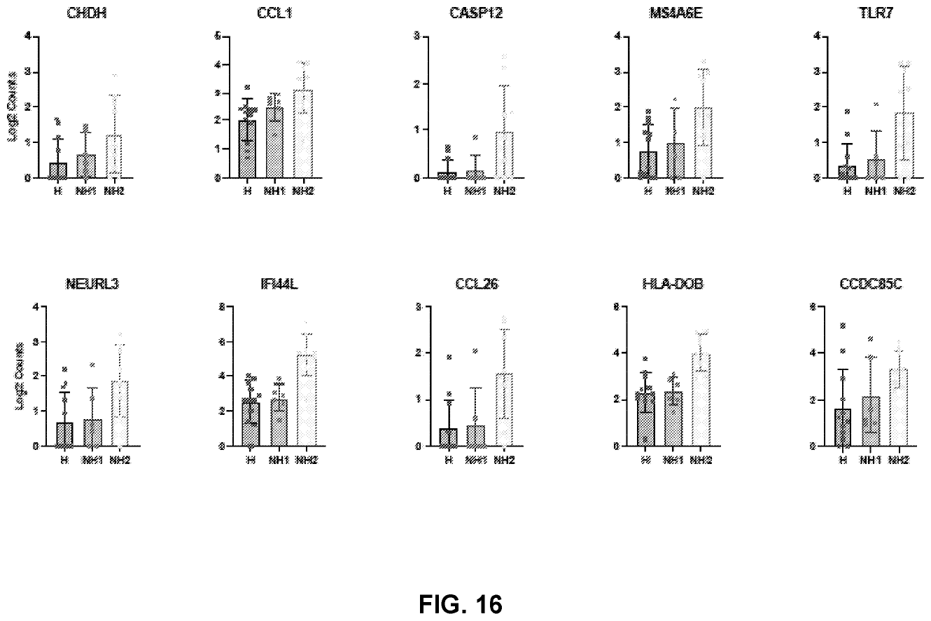

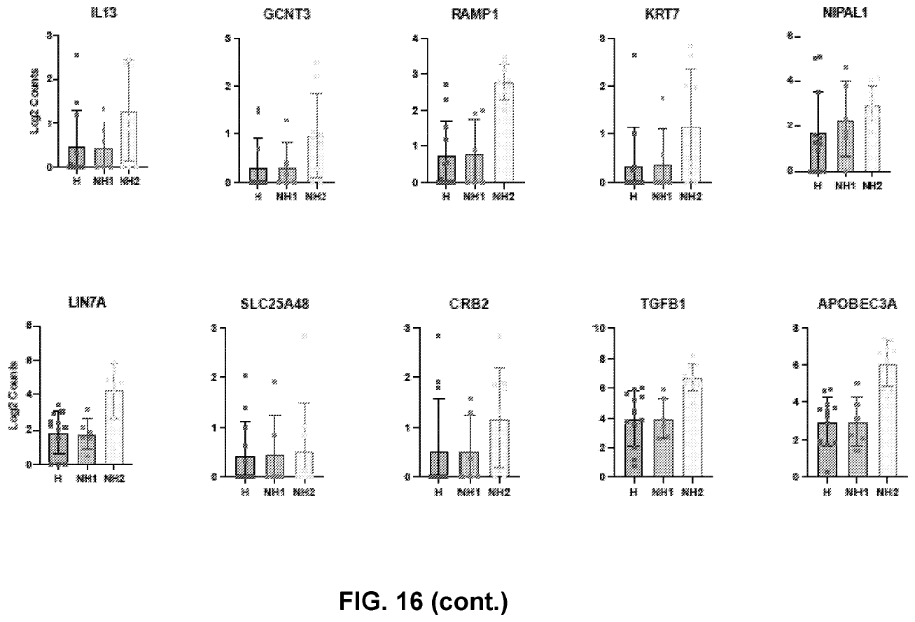

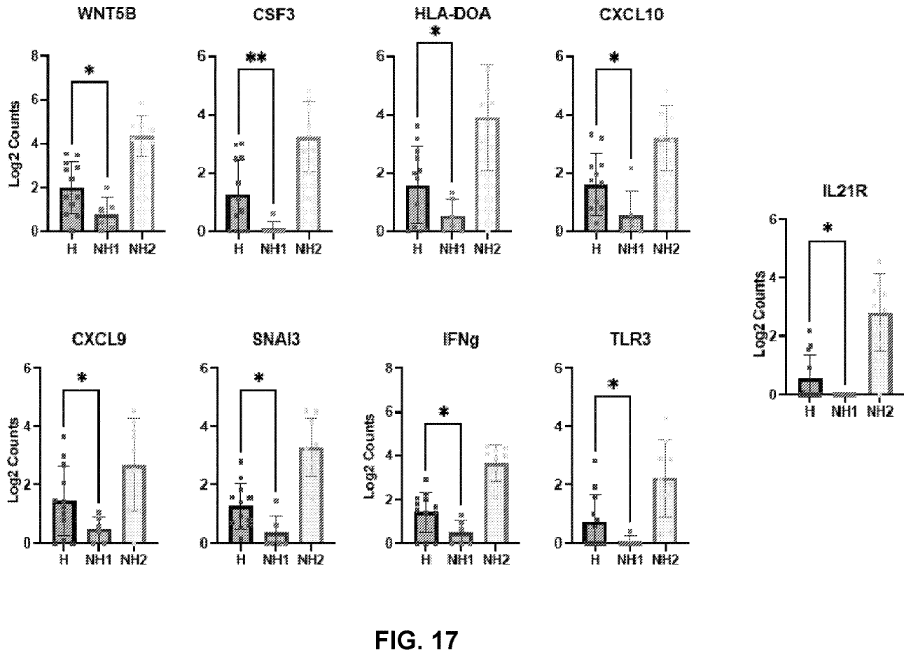

Because the informative genes did not substantially differ between nonhealing subtype 1 and healing subjects, PLS-DA analysis was performed on only those two groups. PLS-DA of healing vs. non-healing subtype 1 wounds showed moderate separation (FIG. 15C). The top 20 genes informing differences between the groups (FIG. 15D) were generally expressed at higher levels by non-healing subtype 1 wounds compared to healing wounds. Most were related to the M2a phenotype (CHDH, MS4A6E, CCL26, CCDC85C, IL13, GCNT3, RAMP1, NIPAL1, SLC25A48, CRB2). The others were associated with the M1 phenotype (CCL1, NEURL3, IFI44L, HLA-DOB, KRT7), the M2c phenotype (LIN7A), wound healing (CASP12), angiogenesis (TGFB1), and wound healing (CASP12). However, none of these genes were statistically significant in their differences between groups (FIG. 16). Therefore, t-tests were performed between the healing and non-healing subtype 1 groups and identified 16 genes that were significantly different. All of these genes were expressed at lower levels in non-healing subtype 1 wounds compared to healing wounds, and half of them were related to early stages of healing (UBD, IL6, IL3RA, CCL8, CSF3, HLA-DOA, CXCL10, CXCL9), suggesting a hypo-inflammatory profile in non-healing subtype 1 (FIG. 17, top 6 genes in FIG. 15E). In summary, this analysis identified two distinct subtypes of non-healing wounds that appeared either hypo-inflammatory (subtype 1) or hyper-inflammatory (subtype 2).

### Example 4: Changes in Gene Expression Over Time

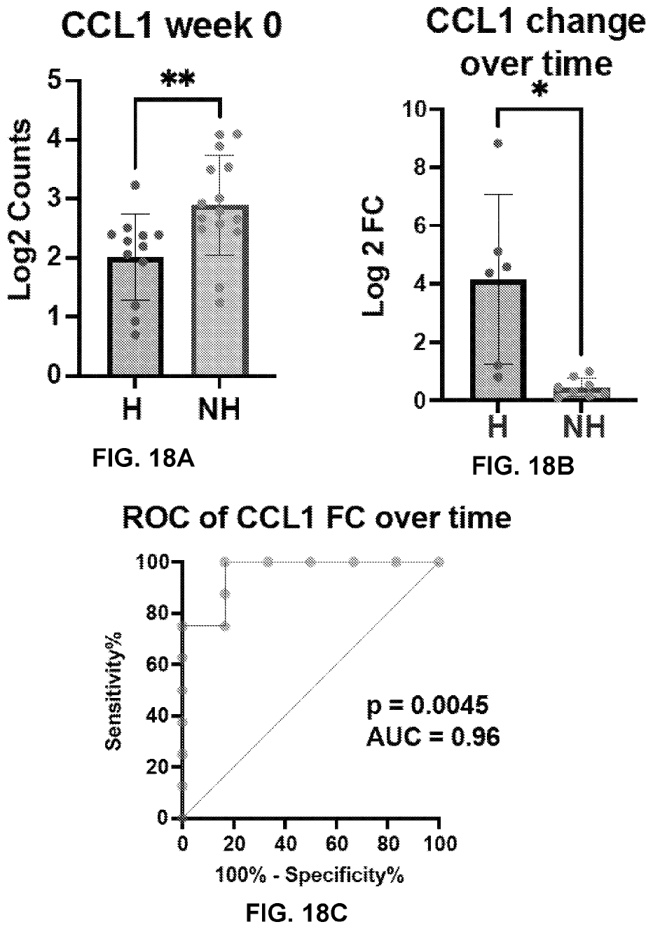

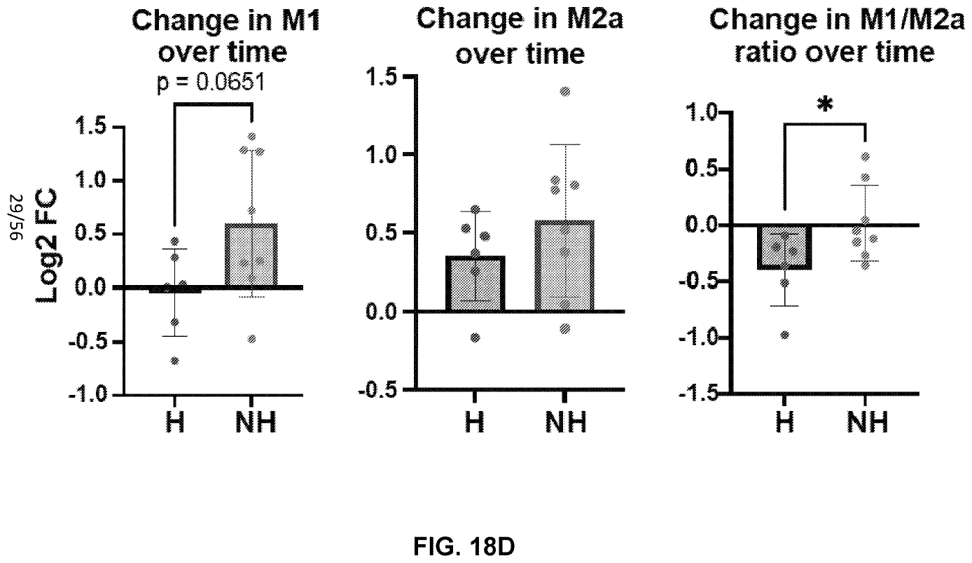

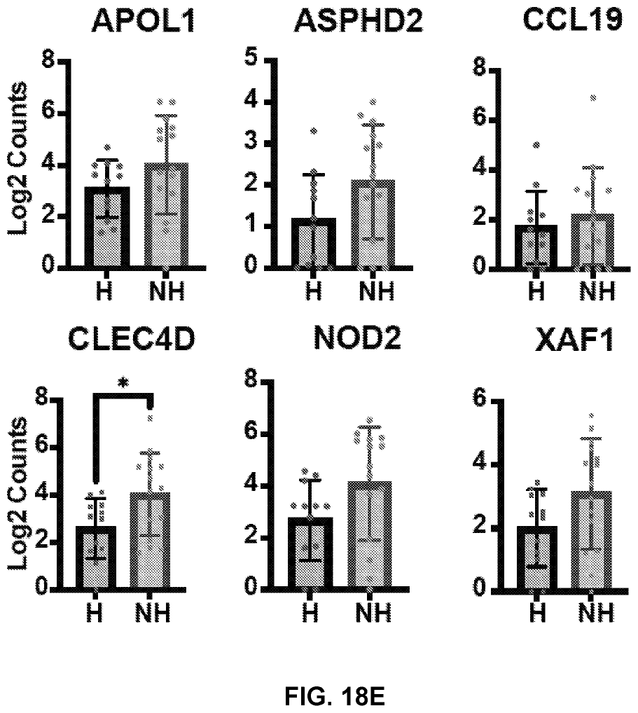

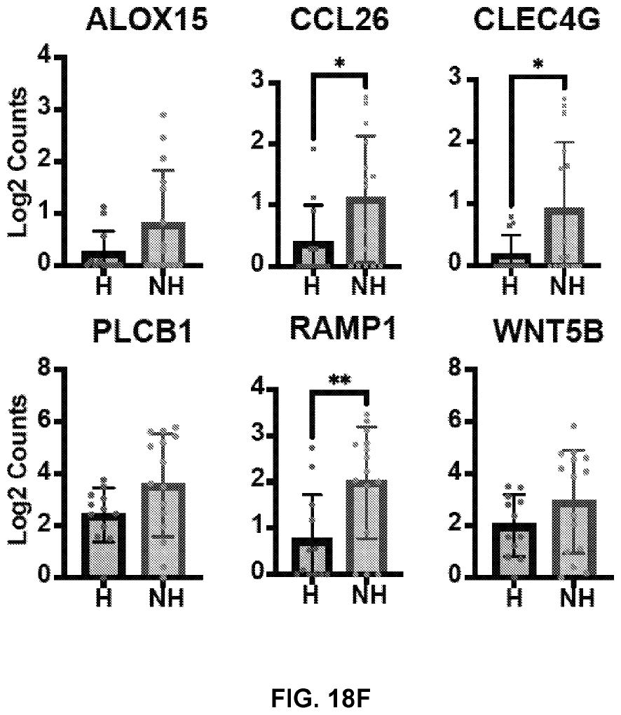

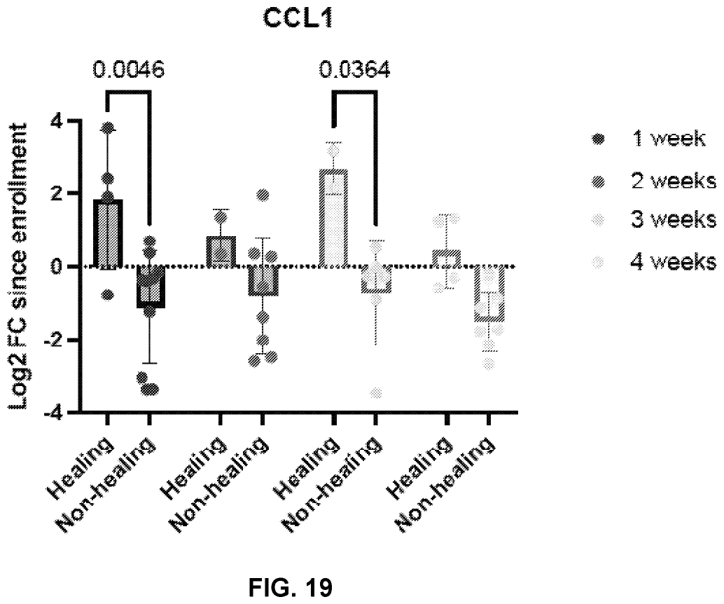

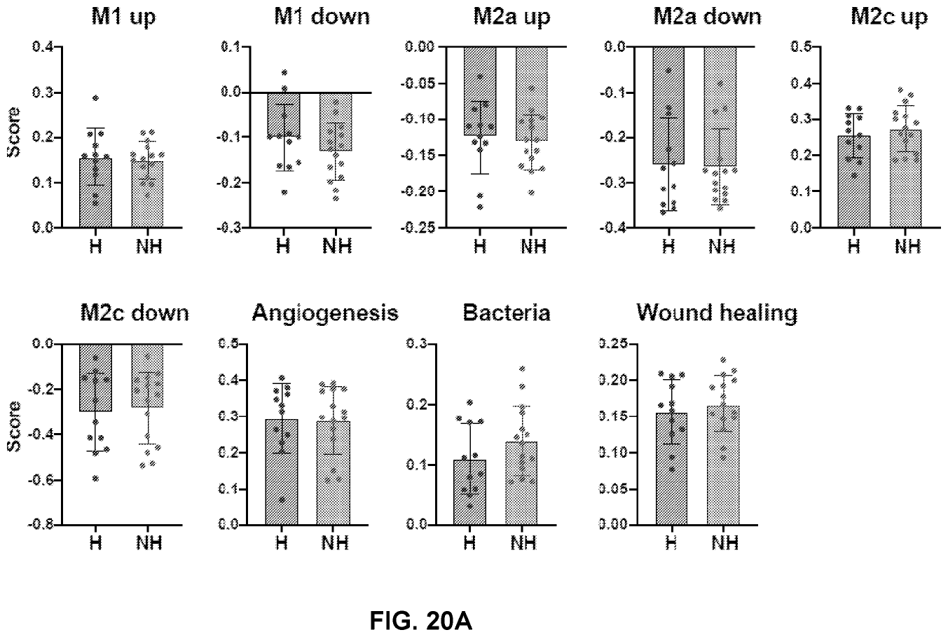

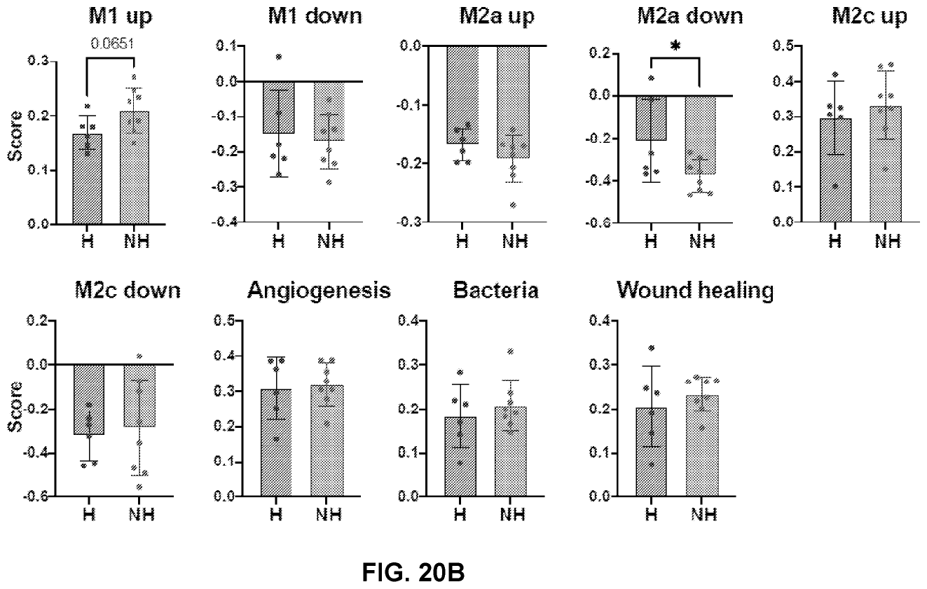

How gene expression changed over time was analyzed in paired samples, and it was found that only CCL1 significantly differed between healing and non-healing DFUs in terms of change compared to week 0. Although CCL1 was initially expressed at higher levels in non-healing wounds (FIG. 18A), CCL1 expression increased in all but one healing DFUs between week 0 and week 4, whereas it decreased in all non-healing DFUs (FIG. 18B), suggesting potential as a biomarker of healing (FIG. 18C). This trend was also observed at weeks 1-3 when compared to week 0 (FIG. 19). It was analyzed how sets of genes changed over this same time period (FIG. 18D). While none of the analyzed gene sets were significantly differentially enriched between groups at week 0 (FIG. 20A), the gene set associated with downregulation of the M2a macrophage phenotype was significantly enriched at week 4 in the healing DFUs compared to non-healing DFUs (FIG. 20B). Enrichment of the M1 macrophage gene set and the ratio between the M1 and M2a gene sets both increased over time to a greater extent in non-healing DFUs compared to healing DFUs (p=0.06 and p=0.05, respectively).

### Example 5: Evaluation of Macrophage-Specific Genes

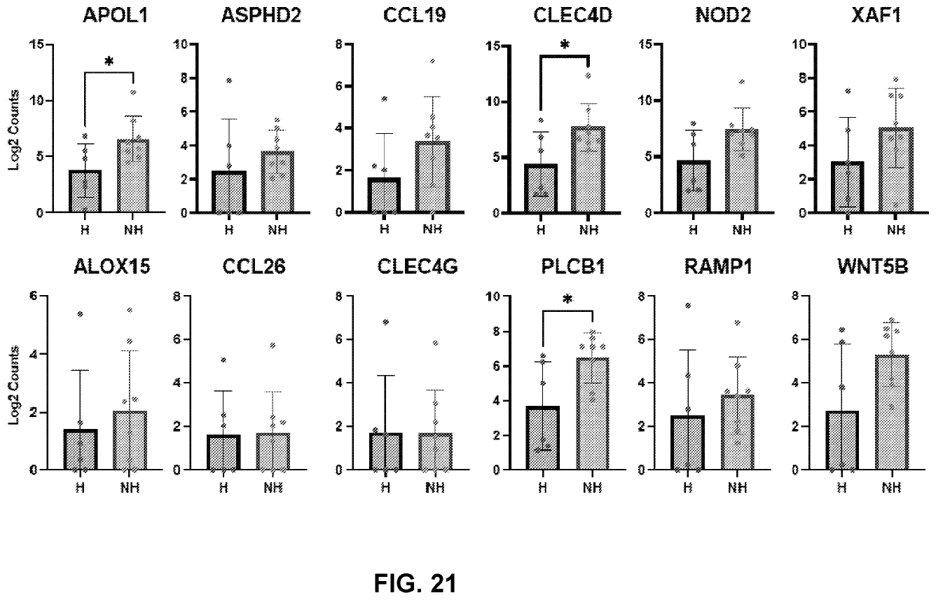

The generally higher expression of inflammation-related genes in non-healing DFUs led to postulation if non-healing DFU tissue contained higher numbers of immune cells, especially macrophages, compared to healing DFUs. Therefore the expression of 12 genes that wsere previously showed to be macrophage-specific (Ferraro, N. M., et al., Integr Biol (Camb) 9, 328-338) was evaluated. In general, all of these genes were expressed at higher levels in non-healing DFUs compared to healing DFUs at both week 0 and week 4 time points, regardless of whether they were associated with M1 or M2a polarization (FIG. 18E, FIG. 18F, FIG. 21), although the effect was more pronounced for genes associated with the M1 phenotype (FIG. 18E). These results suggested that the non-healing DFUs contained more macrophages of both phenotypes than healing DFUs.

### Example 6: Gene Level Analyses after Normalization

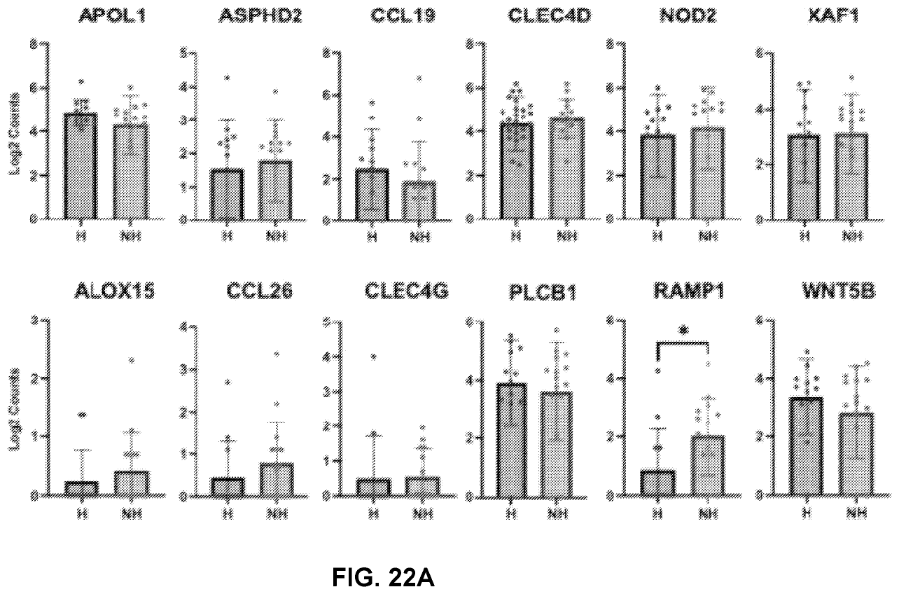

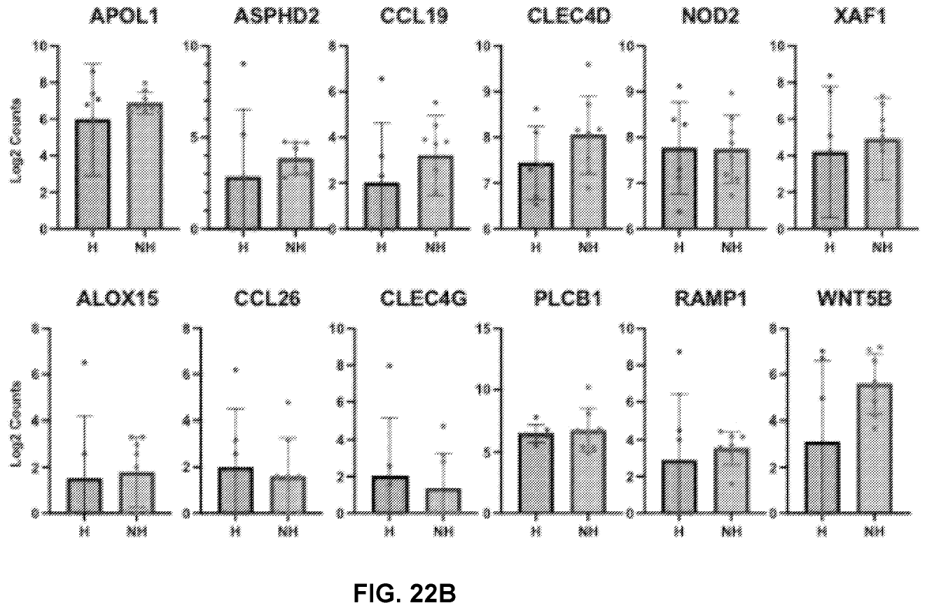

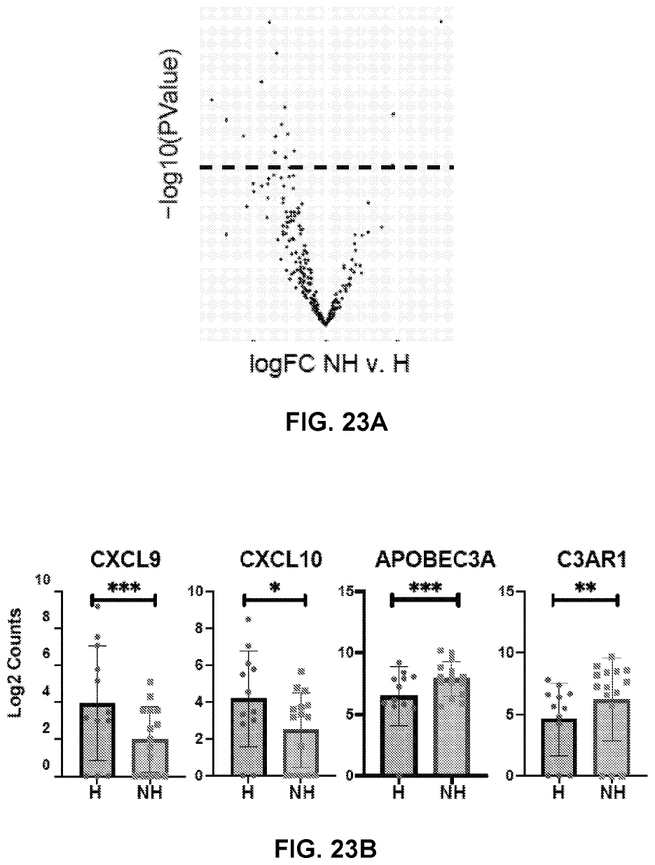

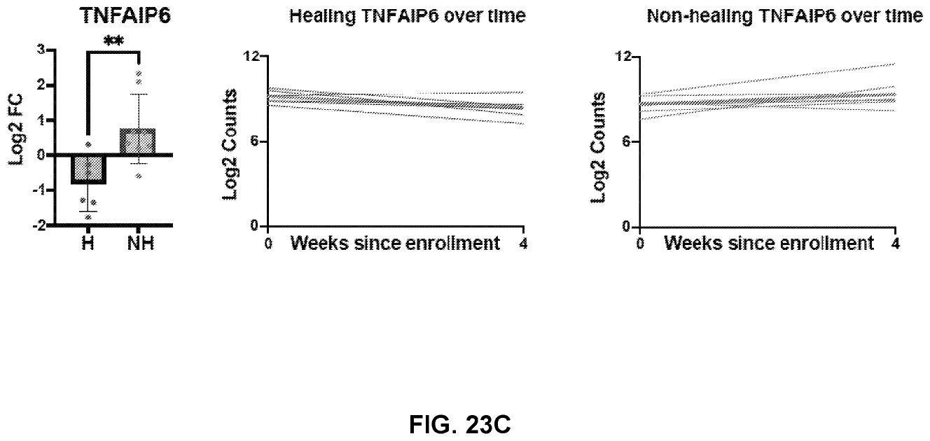

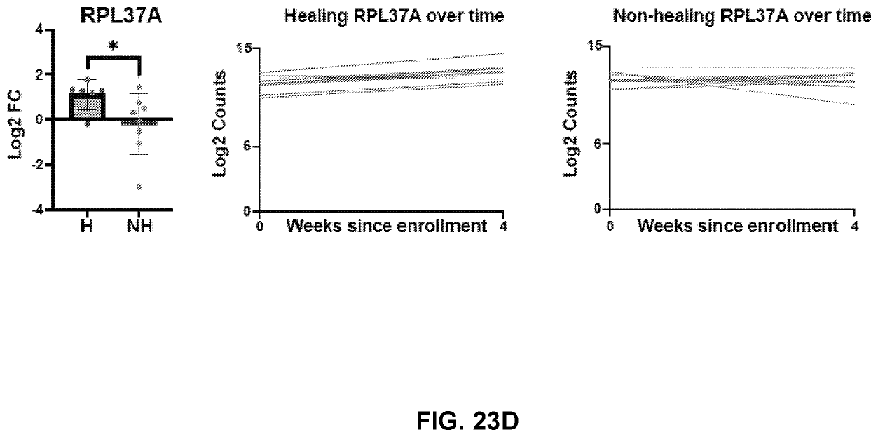

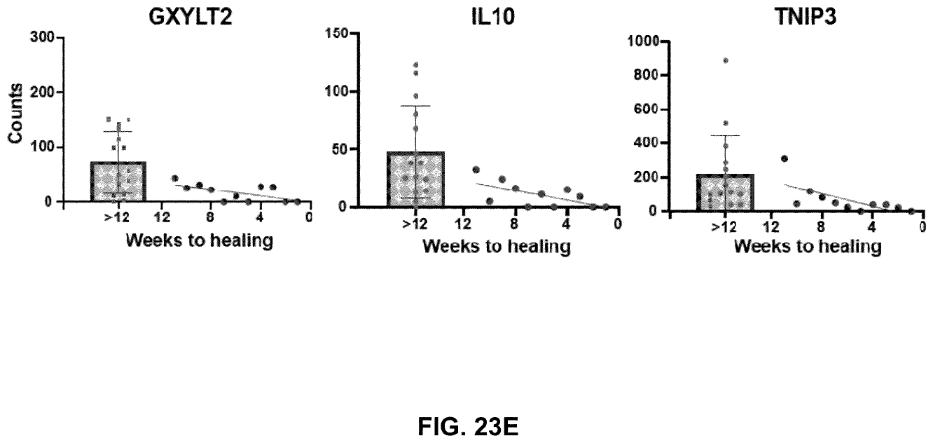

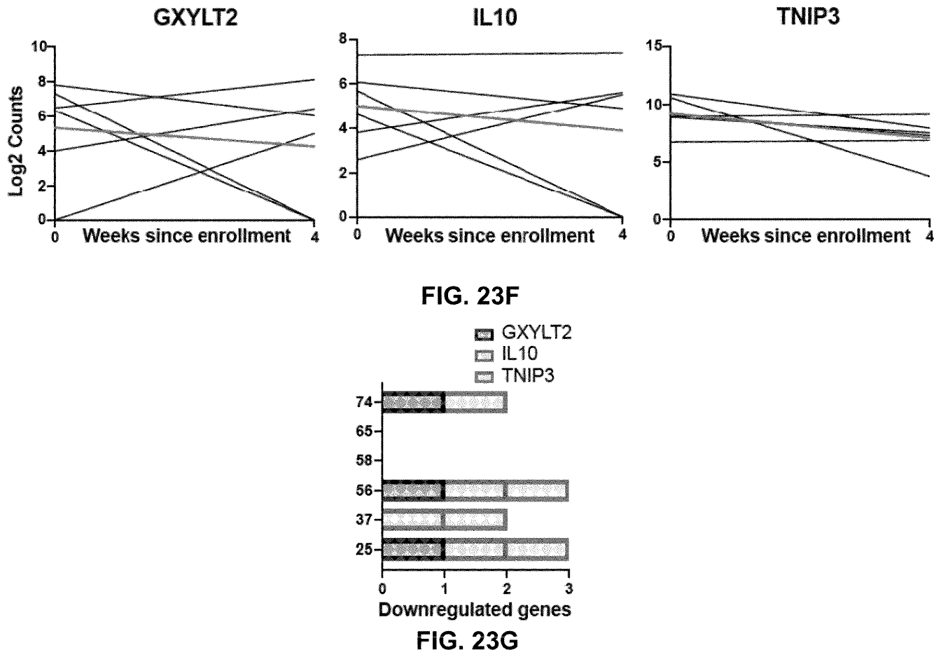

Based on the global increases in gene expression in non-healing DFUs compared to healing DFUs, and especially in macrophage-specific genes, the data were normalized using upper quartile normalization to take these differences in sample composition into account in order to further analyze how cell behavior changes in paired samples over time and how gene expression is influenced by the microbiome. After normalization, macrophage-specific genes were no longer differentially expressed between healing and non-healing DFUs, with the exception of RAMP1, which remained more highly expressed in non-healing DFUs (FIG. 22A-FIG. 22B). In fact only 4 genes were confirmed to be differentially expressed between the groups at p<0.05 and FC>2 (FIG. 23A-FIG. 23B). The two genes expressed at higher levels in healing DFUs, CXCL9 and CXCL10, are associated with the M1 phenotype of macrophages. The other two DEGs were expressed at higher levels in non-healing DFUs and were associated with M1 polarization (APOBEC3A) and host-microbe communication (C3AR1). When evaluating the change in each gene between week 0 and week 4, only 2 genes were found to change to different extents between groups, and they changed in different directions between healing and non-healing DFUs (FIG. 23C). TNFAIP6, an M1 marker, decreased over time in most of the healing DFUs but increased in most of the non-healing DFUs (FIG. 23C). RPL37A, which was included on the panel as a housekeeping gene and is involved in metabolism, generally increased over time in healing DFUs, whereas it increased in some non-healing DFUs and decreased in others (FIG. 23D).

### Example 7: Changes in Expression as a Wound Approaches Healing

All of the healing DFUs in this study healed within 12 weeks of the first sample collection, but at different time points, so whether expression of any gene was associated with the amount of time remaining until complete wound closure across the healing population was investigated. It was found that the expression of three genes, GXYLT2, IL10, and TNIP3, was generally lower for DFUs that were closer to healing (FIG. 23E). While not every subject with paired time points showed decreasing expression of each gene as they progressed towards healing (FIG. 23F), 4 of the 6 patients showed a decrease in at least 2 of these genes over time (FIG. 23G). In contrast, the non-healing DFUs were more variable, with no clear trends compared to healing DFUs.

### Example 8: Relationships Between Human Gene Expression and the Microbiome

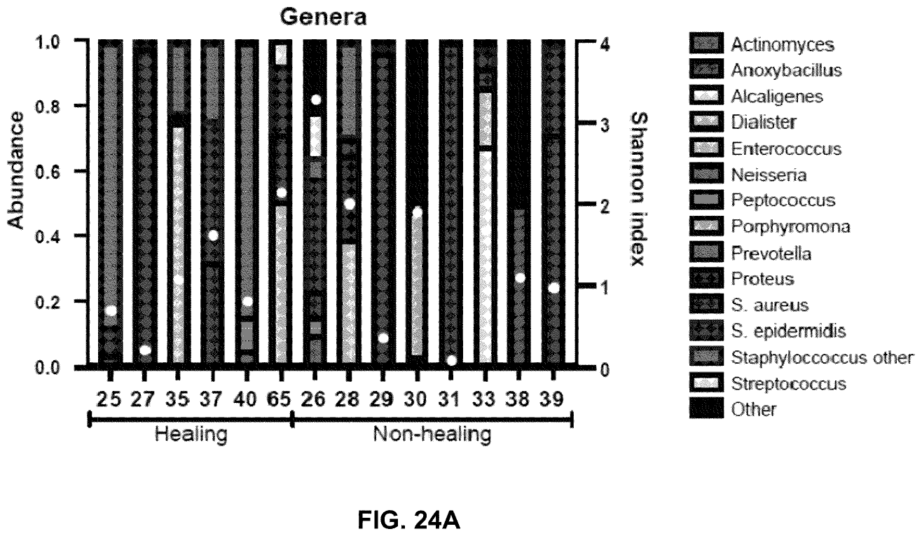

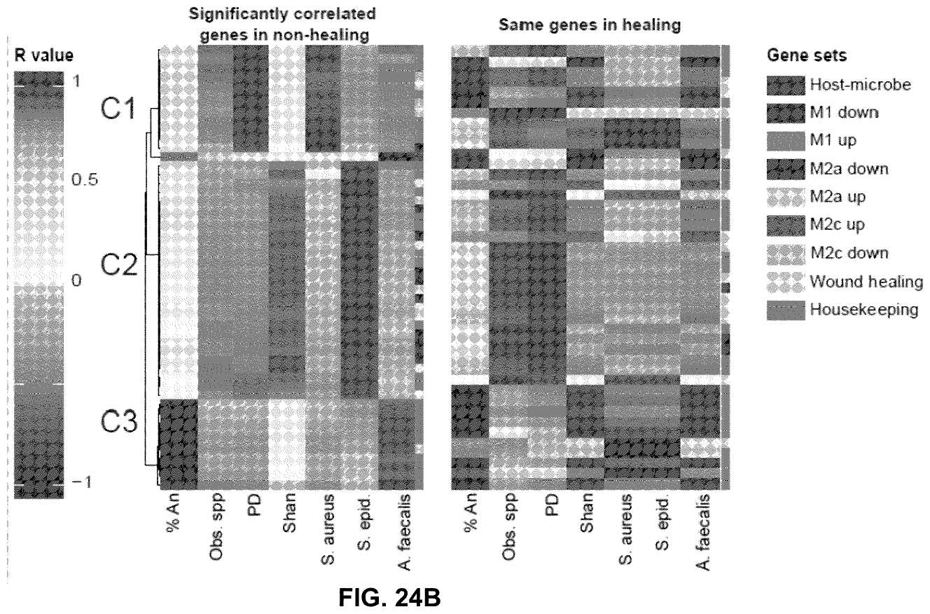

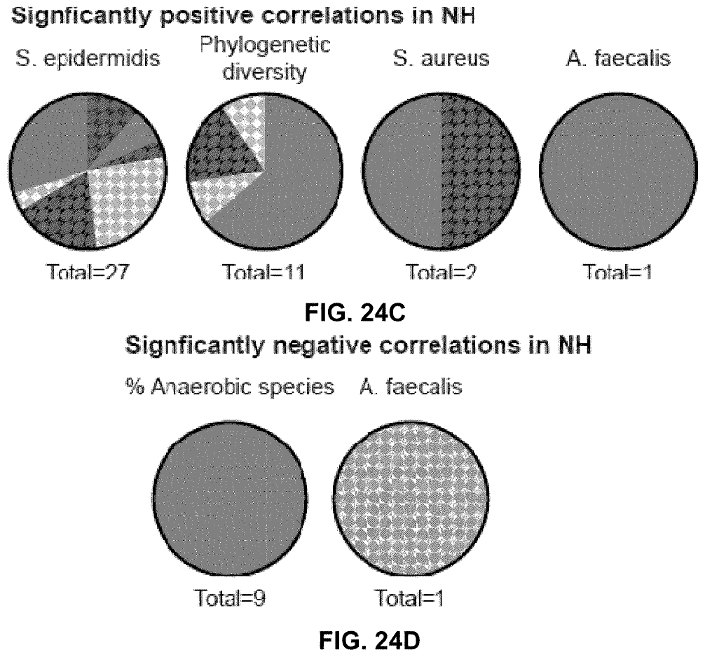

The microbial communities within the DFUs were evaluated as, without wishing to be bound by theory, it was hypothesized that the microbiota influences human tissue gene expression. The microbiome was profiled in a subset of healing (n=6) and non-healing (n=8) DFUs using 16S rRNA gene sequencing at the first time point. Taxonomic composition was heterogenous across DFUs, with prevalent Staphylococcus which was predominant in about half of the DFUs (FIG. 24A). Other wound pathogens, such as Streptococcus and anaerobic bacteria (eg. Prevotella, Porphyromonas, Proteus, Peptococcus) were also identified in the microbiota. Community diversity, measured using the Shannon index, was also variable across healing and non-healing DFUs (FIG. 24A). Significant differences in diversity or microbial composition were not detected in healing vs. non-healing DFUs.

Microbiome variables were next defined to correlate with gene expression values. Three alpha diversity metrics were selected: observed species, Shannon diversity, and phylogenetic diversity (PD). Relative abundance of S. aureus, S. epidermidis, A. faecalis, and aggregated anaerobes was also selected. After codifying the microbiome variables, correlations with gene expression values were analyzed. In the non-healing DFUs, 51 genes were significantly correlated to alpha diversity metrics, but no significant correlations were found in the healing DFUs (FIG. 24B). This finding might be partially explained by the lower number of replicates in this group (n=3 in healing compared to n=6 for non-healing), but even when comparing the same genes in non-healing and healing DFUs there were clear differences in patterns of correlation. For example, within the non-healing DFUs, Cluster 1 (C1) comprised genes that were significantly positively correlated to phylogenetic diversity and to S. aureus relative abundance. These same genes were generally negatively correlated with the same metrics in healing DFUs. Cluster 2 (C2) contained about half of all genes with significant correlations, although no particular gene set was overrepresented in this cluster. In the non-healing DFUs, C2 comprised genes that were positively correlated with S. epidermidis relative abundance and weakly negatively correlated with the number of observed species, phylogenetic diversity, Shannon index, and relative abundance of S. aureus. In contrast, in the healing DFUs, these genes were positively correlated with the number of observed species and phylogenetic diversity, and negatively correlated with Shannon index and relative abundances of S. aureus, and S. epidermis. Finally, Cluster 3 (C3) in the non-healing DFUs, which was primarily M1 markers, was negatively correlated with the percentage of anaerobic species and positively correlated to A. faecalis. These trends were similar in healing DFUs.

Across all clusters, 20 of the 51 significantly correlated genes were associated with M1 macrophage polarization (FIG. 24C). Interestingly, the gene set with the second greatest number of correlations was typical housekeeping genes in which S. epidermidis relative abundance was significantly positively correlated to all 8 of them. Additionally, there were considerably more positive than negative correlations overall and the majority of the genes were correlated to S. epidermidis, which is normally considered a commensal organism (FIG. 24C). Many of these genes were related to regulation of M2a polarization.

### Example 9: Data Normalization

In the present example, the same data set as used for Examples 1-8 above were used to assess the manner in which data normalization techniques influenced the results. Eight different normalization techniques were used to determine how normalization technique influenced the results in terms of differentially expressed genes (DEGs) identified between healing and non-healing groups. These techniques included z-score, percentile, quartile, quantile, total counts, housekeeping genes, or meanshift normalization methods.

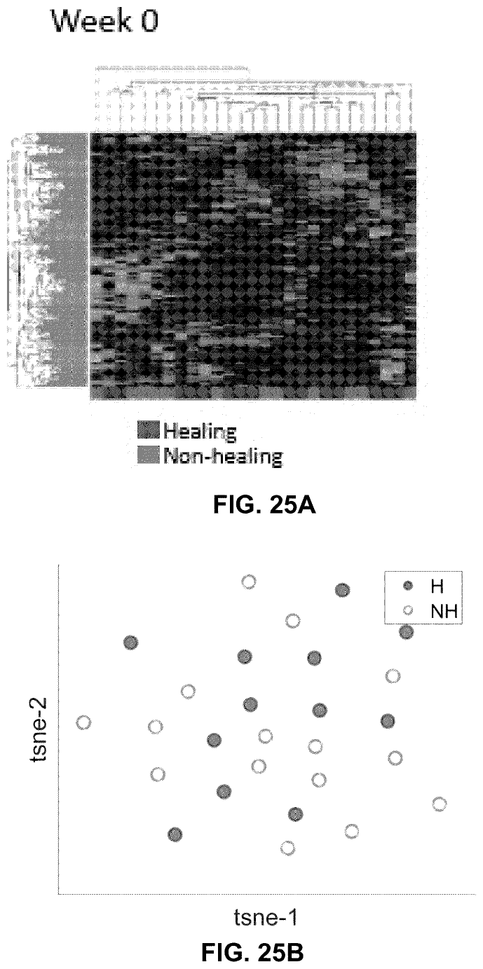

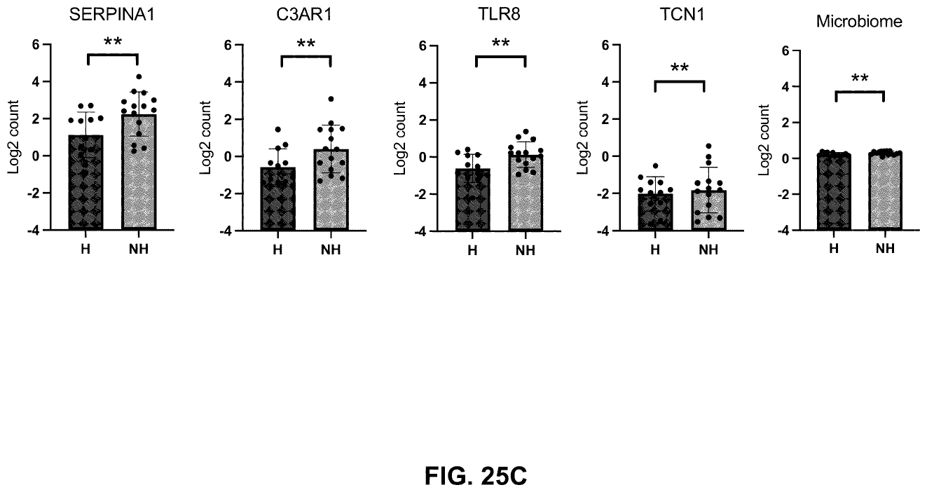

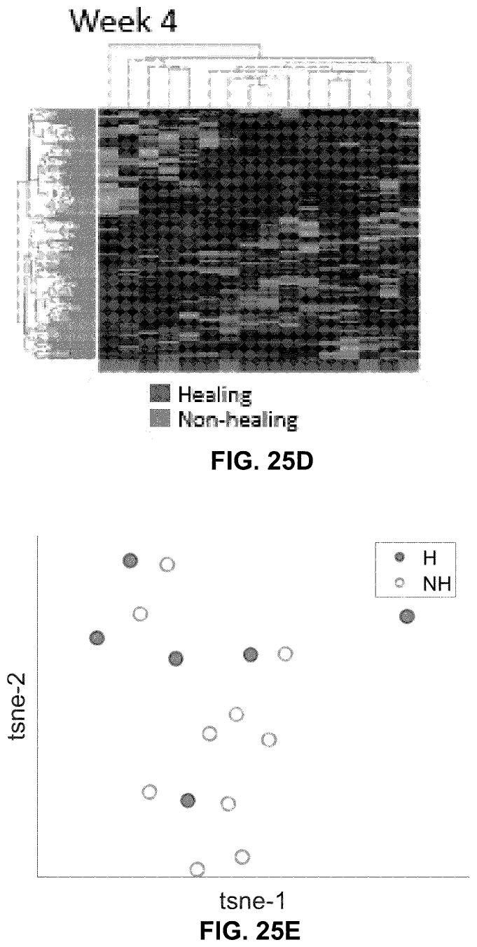

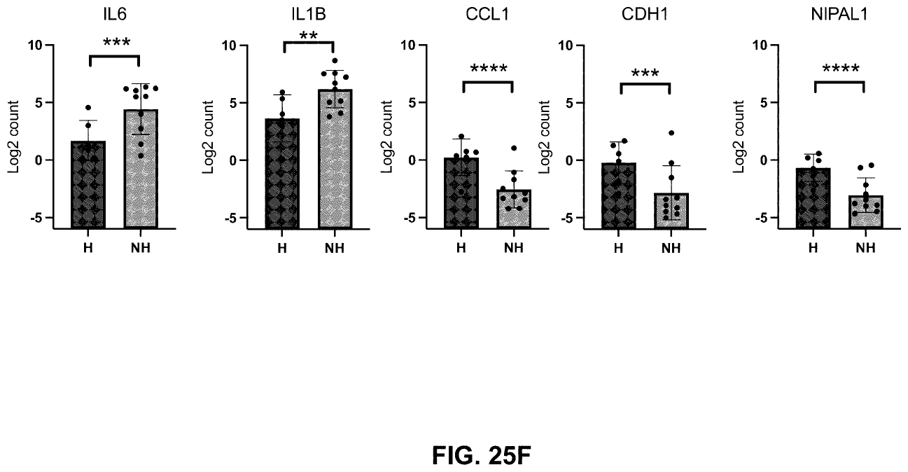

After using the Fisher method to combine p-values across all 8 normalization methods, there were 4 DEGs and 1 differentially expressed gene set at week 0, and 61 DEGs or gene sets at week 4. Hierarchical clustering and multidimensionality reduction (tSNE) of meanshift-normalized data at week 0 showed no clear clustering between healing and non-healing groups (FIG. 25A and FIG. 25B). SERPINA1, C3AR1, TLR8, TCN1, and the Microbiome gene set were differentially expressed at higher levels in the non-healing group compared to the healing group art week 0 (FIG. 25C). At the week 4 time point, the healing and non-healing groups clusters slightly further apart, although the separation between them was still not clear (FIG. 25D and FIG. 25E). The top DEGs at week 4 were IL6 and IL1B, which were both expressed at higher levels in non-healing compared to healing DFUs, and CCL1, CDH1, and NIPAL1, which were expressed at lower levels in non-healing compared to healing DFUs (FIG. 25F).

### Example 10: Changes in Gene Expression Over Time

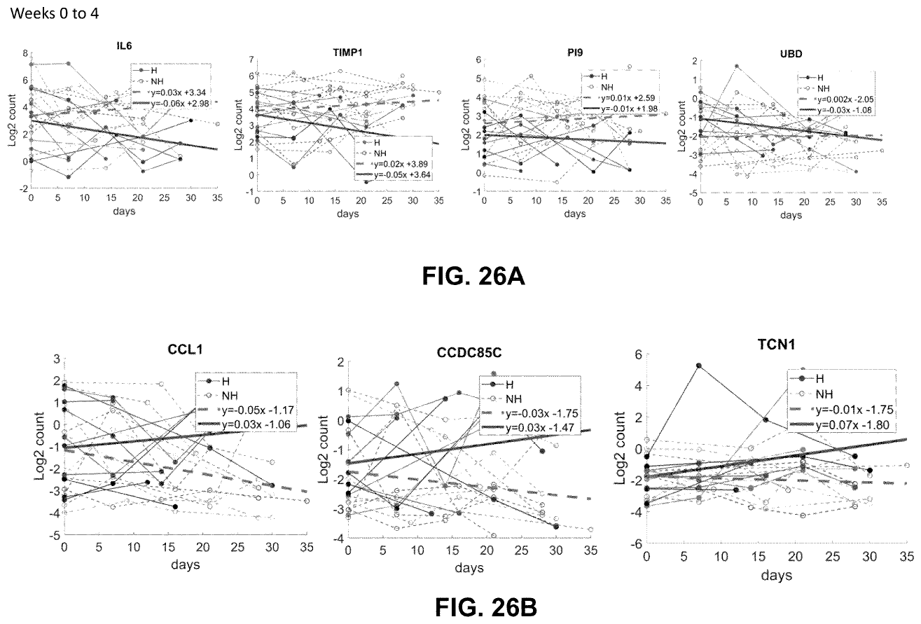

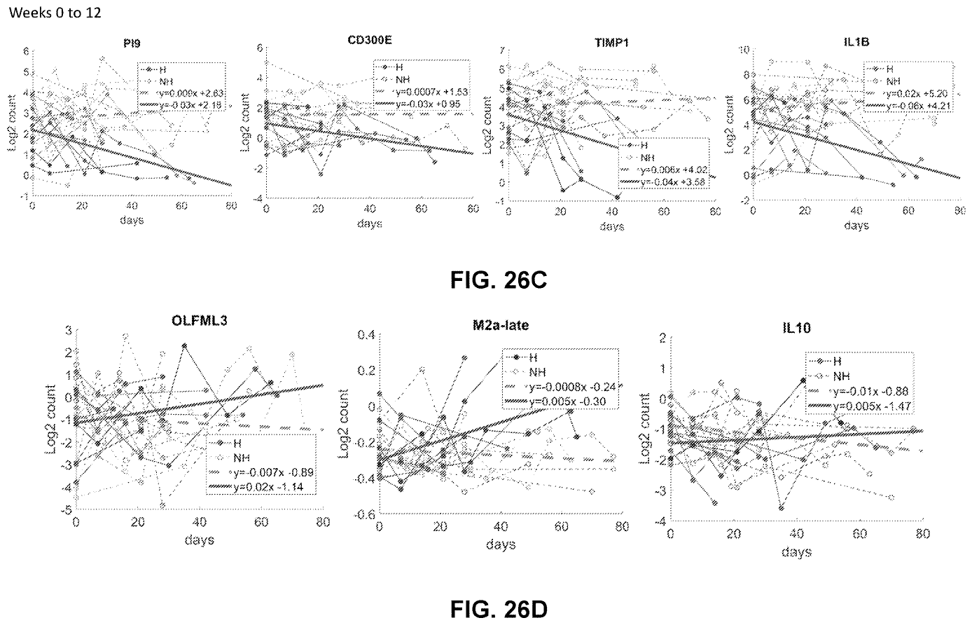

How gene expression changed over time was analyzed and genes whose slopes significantly differed between healing and non-healing subjects were identified (FIG. 26A-FIG. 26D). Expression of IL6, TIMP1, PI9, and UBD generally increased over time for non-healing subjects but decreased over time for healing subjects, while expression of CCL1, CCDC85C, and TCN1 generally decreased over time for non-healing subjects but increased over time for healing subjects.

When analyzing samples collected over 12 weeks from initial sample collection, PI9, CD300E, TIMP1, and ILIB generally increased over time for non-healing subjects but decreased for healing subjects, while OLFML3, the M2a-late stage gene set, and IL10 increased over time for healing subjects but decreased over time for non-healing subjects (FIG. 26C).

### Example 11: Relationships Between Human Gene Expression and the Microbiome

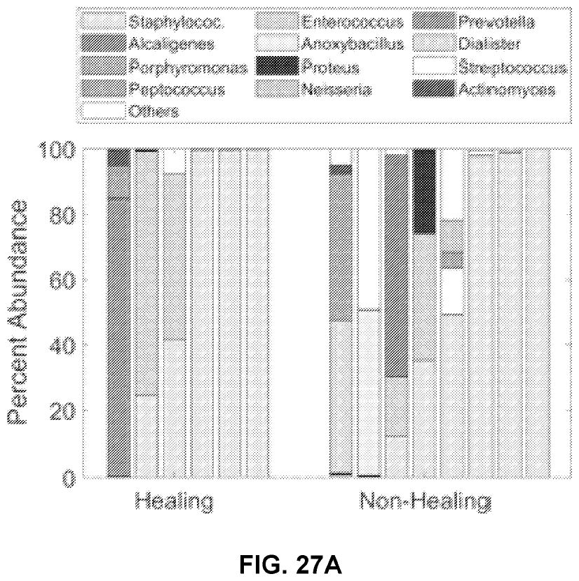

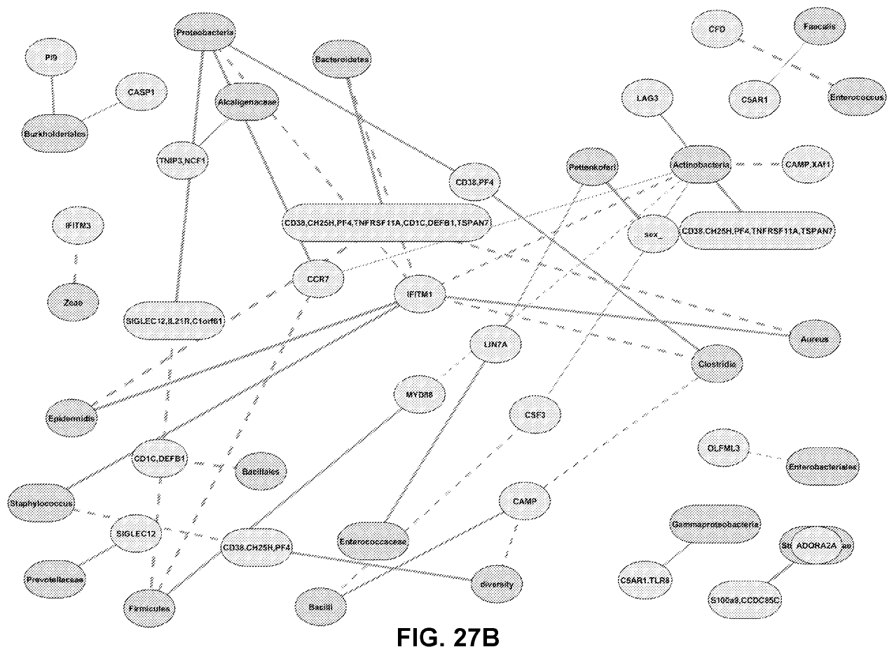

The microbial communities within the DFUs were evaluated as, without wishing to be bound by theory, it was hypothesized that the microbiota influences human tissue gene expression. The microbiome was profiled in a subset of healing (n=6) and non-healing (n=8) DFUs using 16S rRNA gene sequencing at the first time point. Taxonomic composition was heterogenous across DFUs, with prevalent Staphylococcus which was predominant in almost all of the DFUs (FIG. 27A). Other wound pathogens, such as Streptococcus and anaerobic bacteria (eg. Prevotella, Porphyromonas, Proteus, Peptococcus) were also identified in the microbiota. Community diversity, measured using the Shannon index, was also variable across healing and non-healing DFUs. Significant differences in microbial composition were not detected, and diversity in healing vs. non-healing DFUs was not detected.

Correlations between microbial species or diversity metrics and human gene expression values were analyzed using samples collected at week 0.

While 30 genes were found to be significantly correlated with the microbiome of healing DFUs, only 6 genes were found to be significantly correlated with the microbiome in non-healing DFUs (FIG. 27B). No genes were found to be correlated with the microbiome in both healing and non-healing DFUs, highlighting a lack of similarity in crosstalk with the microbiome between the two groups.

### Example 12: Identification of Most Predictive Genes

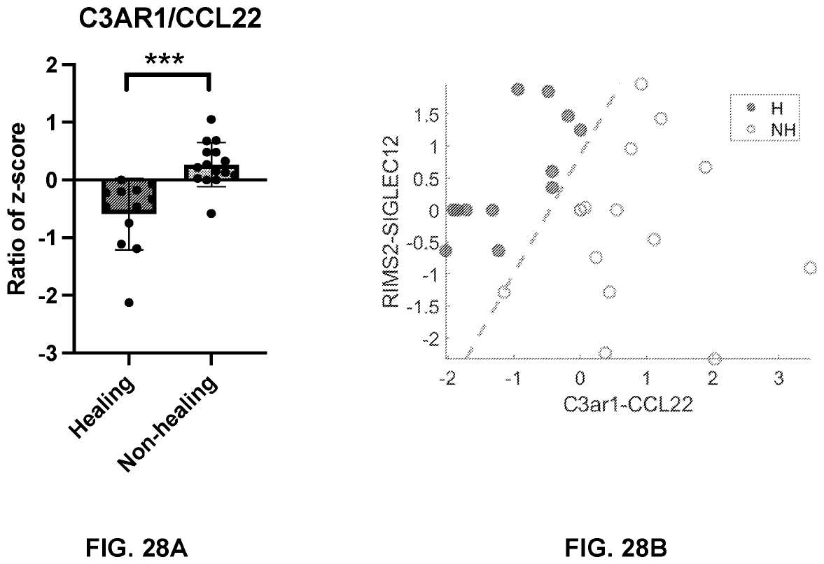

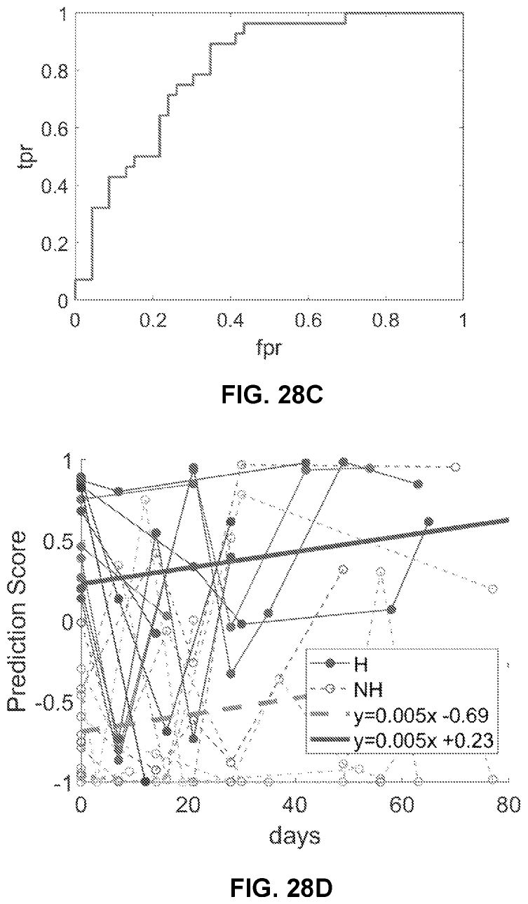

Finally, machine learning was used to identify if any genes could be used alone or in combination to accurately predict healing outcome when using samples collected at the week 0 time points only, which would be useful as a clinical biomarker to facilitate decision making about how to care for these patients. In addition to individual genes, all possible pairs of genes were analyzed when processed as a ratio, because previous work showed that tracking ratios of pro-inflammatory to reparative genes over time could be used to distinguish between healing and non-healing subjects (Nassiri, S., et al., The Journal of investigative dermatology 135, 1700-1703 (2015)). While no individual genes accurately distinguished between healing outcome, there were several combinations of gene ratios that could be used to predict healing outcome with 100% accuracy, especially when combined multiple other gene ratios. For example, the ratio of C3AR1 to CCL22 at week 0 almost perfectly separated healing and non-healing subjects, with only 2 of 27 samples misclassified when using a log-fold change in z-score of 0 or higher as non-healing and less than 0 as healing (FIG. 28A). When combined with the ratio of RIMS2 to SIGLEC12 in a logistic regression model, all samples collected at week 0 were accurately classified as healing and non-healing (FIG. 28B). This model was then tested for accuracy of prediction across all samples collected between weeks 0 and 12 (FIG. 28C and FIG. 28D). The model was more accurate for samples collected at later time points compared to earlier time points (FIG. 28D).

### ENUMERATED EMBODIMENTS

The following enumerated embodiments are provided, the numbering of which is not to be construed as designating levels of importance.

Embodiment 1 provides a method of treating and/or ameliorating a non-healing wound in a subject in need thereof, the method comprising measuring the levels of expression of a first panel of inflammation-related genes from a sample from the wound, and measuring the levels of expression of a second panel of inflammation-related genes from the sample, wherein when at least one gene from the first panel or a composite score of genes from the first panel or a ratio compared to genes from the second panel is expressed at a lower level than a reference sample, a pro-inflammatory agent is administered to the subject, and wherein when at least one gene from the second panel or a composite score of genes from the second panel or a ratio compared to genes from the first panel is expressed at a higher level than a reference sample, an anti-inflammatory or M2-promoting agent is administered to the subject, thus treating and/or ameliorating the wound in the subject.

Embodiment 2 provides the method of embodiment 1, wherein the first panel and/or the second panel comprises RIMS2, CXCL11, EBI3, ST8SIA6, IFNG, IL6, IL3RA, FCGR2B, TLR2, SPP1, IL15, and TNFRSF1B.

Embodiment 3 provides the method of embodiment 1 or embodiment 2, wherein the first panel and/or the second panel comprises ADORA2A, ANKRD22, APOBEC3A, APOL1, ASPHD2, C1ORF61, CASP1, CCL1, CCL19, CCL5, CCL8, CCR7, CD38, CD80, CFB, CFH, CLEC4D, CLEC4E, CMPK2, CRISPLD2, CSF3, CXCL10, CXCL11, CXCL9, EBI3, EPHA2, FBXO2, GBP1, GBP4, GBP5, GCH1, HAPLN3, HCAR3, HLA-DOA, HLA-DOB, HSH2D, IDO1, IDO2, IFI44L, IFITM1, IFITM3, IGFBP4, IL15, IL15RA, ILIB, IL27, IL32, IL3RA, IL6, IL8, IRF1, ISG15, ISG20, ITK, KRT7, LAG3, MN1, MTIM, NCF1, NCF1B, NEURL3, NNMT, NOD2, OASL, PDE4B, PRSS8, PTGES, PTGS2, RIMS2, RSAD2, SERPINB7, SERPING1, TNFAIP6, TNIP3, UBD, VEGFA, XAF1, and ZBP1.

Embodiment 4 provides the method of any one of the preceding embodiments, wherein the ratio compared to genes from the first panel or the ratio compared to genes from the second panel is a ratio of C3AR1 to CCL22, a ratio of RIMS2 to SIGLEC12, or a combination of a ratio of C3AR1 to CCL22 and a ratio of RIMS2 to SIGLEC12

Embodiment 5 provides a method of treating and/or ameliorating a non-healing wound in a subject in need thereof, the method comprising: measuring the levels of expression of a first panel of inflammation-related genes from a sample from the wound, and measuring the levels of expression of a second panel of M2 macrophage-related genes from the sample, wherein when the sample is classified by a previously-trained machine-learning algorithm as hypo-inflammatory, a pro-inflammatory agent is administered to the subject, and wherein when the sample is classified by a previously-trained machine-learning algorithm as hyper-inflammatory, an anti-inflammatory or M2-promoting agent is administered to the subject thus treating and/or ameliorating the wound in the subject.

Embodiment 6 provides the method of any one of the preceding embodiments, wherein the second panel of M2 macrophage-related genes comprises ABCG2, ALDH1A2, ALOX15, ATOH8, CABLES1, CACNA1G, CACNB4, CCL22, CCL26, CCL28, CCDC85C, CD1C, CDH1, CH25H, CHDH, CLEC4G, COL5A3, CR2, CRB2, DACT1, DNASE1L3, DUOX1, DUOXA1, EHF, ENHO, FABP4, FAM110B, FCGR2B, FOXQ1, GCNT3, IL17RB, IL21R, LIMA1, LRRC4, MAOA, MEST, MORC4, MRC1, MS4A6E, MUCL1, NEK10, NIPAL1, OLFML3, PALD1, PCSK1, PDGFB, PLCB1, PLEKHA6, RAMP1, S100A1, SEMA3G, SIGLEC12, SLC25A48, SNAI3, ST8SIA6, SYT17, TAL1, TGM2, TIMP3, TNFRSF11A, TSPAN7, VTN, WDR66, WNT5B.

Embodiment 7 provides the method of any one of the preceding embodiments, wherein the pro-inflammatory agent is selected from the group consisting of a glutaraldehyde-crosslinked material, a xenogeneic protein-derived material, a skin regeneration system, an acellular dermal matrix, a biologic extracellular matrix, biomaterials that promote the M1 phenotype in host macrophages through the release of drugs, proteins, or nucleic acids, and M1 macrophage cell therapy.

Embodiment 8 provides the method of any one of the preceding embodiments, wherein the anti-inflammatory or M2-promoting agent is selected from the group consisting of an amniotic membrane-derived tissue, a placental-derived tissue, a bioengineered allogeneic cellular construct, an extracellular matrix-derived material, urinary bladder matrix (UBM), biomaterials that promote the M2 phenotype in host macrophages through the release of drugs, proteins, or nucleic acids, and M2 macrophage cell therapy.

Embodiment 9 provides the method of any one of the preceding embodiments, wherein the wound is an ulcer.

Embodiment 10 provides the method of any one of the preceding embodiments, wherein the wound is a diabetic ulcer.

Embodiment 11 provides the method of any one of the preceding embodiments, wherein the sample is obtained by swabbing the wound, or debriding the wound and collecting the debrided tissue.

Embodiment 12 provides the method of any one of the preceding embodiments, wherein expression is measured at the mRNA level or protein level.

Embodiment 13 provides the method of any one of the preceding embodiments, further comprising administering an additional treatment.

Embodiment 14 provides the method of embodiment 13, wherein the additional treatment is selected from the group consisting of debriding the wound, applying a compression wrapping, applying a compression stocking, applying dressings promoting a moist environment to the wound, applying a wound offloading device, applying a hyperbaric oxygen therapy, applying an antibiotic, administering an immunomodulation medication, or combinations thereof.

Embodiment 15 provides the method of any one of the preceding embodiments, wherein the subject is a human.

Embodiment 16 provides a method of treating and/or ameliorating a non-healing wound in a subject in need thereof, the method comprising: measuring the levels of expression of a first panel of inflammation-related genes from a sample from the wound, and measuring the levels of expression of a second panel of inflammation-related genes from the sample, wherein when the sample is classified by a previously-trained machine-learning algorithm as hypo-inflammatory, a pro-inflammatory agent is administered to the subject, and wherein when the sample is classified by a previously-trained machine-learning algorithm as hyper-inflammatory, an anti-inflammatory or M2-promoting agent is administered to the subject, thus treating and/or ameliorating the wound in the subject.

Embodiment 17 provides a method of treating and/or ameliorating a non-healing wound in a subject in need thereof, the method comprising: measuring the levels of expression of a panel of M2 macrophage-associated genes in a subject, wherein when the sample is classified by a previously-trained machine-learning algorithm as hyper-inflammatory or having too little M2 macrophage-associated gene expression, an anti-inflammatory or M2-promoting agent is administered to the subject, thus treating and/or ameliorating the wound in the subject.

Embodiment 18 provides the method of any one of embodiments 5-17, wherein the previously-trained machine-learning algorithm is a Partial Least-Squares Discriminant Analysis (PLS-DA) algorithm, support vector machine, or neural network.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A method of treating and/or ameliorating a non-healing wound in a subject in need thereof, the method comprising:
measuring the levels of expression of a first panel of inflammation-related genes from a sample from the wound, and
measuring the levels of expression of a second panel of inflammation-related genes from the sample,
wherein when at least one gene from the first panel or a composite score of genes from the first panel or a ratio compared to genes from the second panel is expressed at a lower level than a reference sample, a pro-inflammatory agent is administered to the subject, and
wherein when at least one gene from the second panel or a composite score of genes from the second panel or a ratio compared to genes from the first panel is expressed at a lower level than a reference sample, an anti-inflammatory or M2-promoting agent is administered to the subject,
thus treating and/or ameliorating the wound in the subject.

2. The method of claim 1, wherein the first panel and/or the second panel comprises RIMS2, CXCL11, EBI3, ST8SIA6, IFNG, IL6, IL3RA, FCGR2B, TLR2, SPP1, IL15, and TNFRSF1B.

3. The method of claim 1, wherein the first panel and/or the second panel comprises ADORA2A, ANKRD22, APOBEC3A, APOL1, ASPHD2, C1ORF61, CASP1, CCL1, CCL19, CCL5, CCL8, CCR7, CD38, CD80, CFB, CFH, CLEC4D, CLEC4E, CMPK2, CRISPLD2, CSF3, CXCL10, CXCL11, CXCL9, EBI3, EPHA2, FBXO2, GBP1, GBP4, GBP5, GCH1, HAPLN3, HCAR3, HLA-DOA, HLA-DOB, HSH2D, IDO1, IDO2, IFI44L, IFITM1, IFITM3, IGFBP4, IL15, IL15RA, ILIB, IL27, IL32, IL3RA, IL6, IL8, IRF1, ISG15, ISG20, ITK, KRT7, LAG3, MN1, MTIM, NCF1, NCF1B, NEURL3, NNMT, NOD2, OASL, PDE4B, PRSS8, PTGES, PTGS2, RIMS2, RSAD2, SERPINB7, SERPING1, TNFAIP6, TNIP3, UBD, VEGFA, XAF1, and ZBP1.

4. The method of claim 1, wherein the ratio compared to genes from the first panel or the ratio compared to genes from the second panel is a ratio of C3AR1 to CCL22, a ratio of RIMS2 to SIGLEC12, or a combination of a ratio of C3AR1 to CCL22 and a ratio of RIMS2 to SIGLEC12.

5. A method of treating and/or ameliorating a non-healing wound in a subject in need thereof, the method comprising:
measuring the levels of expression of a first panel of inflammation-related genes from a sample from the wound, and
measuring the levels of expression of a second panel of M2 macrophage-related genes from the sample,
wherein when the sample is classified by a previously-trained machine-learning algorithm as hypo-inflammatory, a pro-inflammatory agent is administered to the subject, and
wherein when the sample is classified by a previously-trained machine-learning algorithm as hyper-inflammatory, an anti-inflammatory or M2-promoting agent is administered to the subject,
thus treating and/or ameliorating the wound in the subject.

6. The method of claim 5, wherein the second panel of M2 macrophage-related genes comprises ABCG2, ALDH1A2, ALOX15, ATOH8, CABLES1, CACNA1G, CACNB4, CCL22, CCL26, CCL28, CCDC85C, CD1C, CDH1, CH25H, CHDH, CLEC4G, COL5A3, CR2, CRB2, DACT1, DNASE1L3, DUOX1, DUOXA1, EHF, ENHO, FABP4, FAM110B, FCGR2B, FOXQ1, GCNT3, IL17RB, IL21R, LIMA1, LRRC4, MAOA, MEST, MORC4, MRC1, MS4A6E, MUCL1, NEK10, NIPAL1, OLFML3, PALD1, PCSK1, PDGFB, PLCB1, PLEKHA6, RAMP1, S100A1, SEMA3G, SIGLEC12, SLC25A48, SNAI3, ST8SIA6, SYT17, TAL1, TGM2, TIMP3, TNFRSF11A, TSPAN7, VTN, WDR66, WNT5B.

7. The method of claim 5, wherein the pro-inflammatory agent is selected from the group consisting of a glutaraldehyde-crosslinked material, a xenogeneic protein-derived material, a skin regeneration system, an acellular dermal matrix, a biologic extracellular matrix, biomaterials that promote the M1 phenotype in host macrophages through the release of drugs, proteins, or nucleic acids, and M1 macrophage cell therapy.

8. The method of claim 5, wherein the anti-inflammatory or M2-promoting agent is selected from the group consisting of an amniotic membrane-derived tissue, a placental-derived tissue, a bioengineered allogeneic cellular construct, an extracellular matrix-derived material, urinary bladder matrix (UBM), biomaterials that promote the M2 phenotype in host macrophages through the release of drugs, proteins, or nucleic acids, and M2 macrophage cell therapy.

9. The method of claim 5, wherein the wound is an ulcer.

10. The method of claim 5, wherein the wound is a diabetic ulcer.

11. The method of claim 5, wherein the sample is obtained by swabbing the wound, or debriding the wound and collecting the debrided tissue.

12. The method of claim 5, wherein expression is measured at the mRNA level or protein level.

13. The method of claim 5, further comprising administering an additional treatment.

14. The method of claim 13, wherein the additional treatment is selected from the group consisting of debriding the wound, applying a compression wrapping, applying a compression stocking, applying dressings promoting a moist environment to the wound, applying a wound offloading device, applying a hyperbaric oxygen therapy, applying an antibiotic, administering an immunomodulation medication, or combinations thereof.

15. The method of claim 5, wherein the subject is a human.

16. A method of treating and/or ameliorating a non-healing wound in a subject in need thereof, the method comprising:
measuring the levels of expression of a first panel of inflammation-related genes from a sample from the wound, and
measuring the levels of expression of a second panel of inflammation-related genes from the sample,
wherein when the sample is classified by a previously-trained machine-learning algorithm as hypo-inflammatory, a pro-inflammatory agent is administered to the subject, and
wherein when the sample is classified by a previously-trained machine-learning algorithm as hyper-inflammatory, an anti-inflammatory or M2-promoting agent is administered to the subject,
thus treating and/or ameliorating the wound in the subject.

17. A method of treating and/or ameliorating a non-healing wound in a subject in need thereof, the method comprising:
measuring the levels of expression of a panel of M2 macrophage-associated genes in a subject,
wherein when the sample is classified by a previously-trained machine-learning algorithm as hyper-inflammatory or having too little M2 macrophage-associated gene expression, an anti-inflammatory or M2-promoting agent is administered to the subject,
thus treating and/or ameliorating the wound in the subject.

18. The method of claim 5, wherein the previously-trained machine-learning algorithm is a Partial Least-Squares Discriminant Analysis (PLS-DA) algorithm, support vector machine, or neural network.
